(19) United States
(12) Patent Application Publication
(10) Pub. No.: US 2024/0315968 A1
(43) Pub. Date: Sep. 26, 2024

(54) SUSTAINED RELEASE CANCER THERAPEUTICS FORMULATIONS

(71) Applicant: inSitu Biologics, Inc., Oakdale, MN (US)

(72) Inventors: William J. Taylor, Woodbury, MN (US); Kelsey Pflepsen, Minneapolis, MN (US)

(21) Appl. No.: 18/615,846

(22) Filed: Mar. 25, 2024

Related U.S. Application Data
(60) Provisional application No. 63/454,507, filed on Mar. 24, 2023.

(51) Int. Cl. A61K 9/107; A61K 9/00; A61K 31/337; A61K 31/704; A61K 45/06; A61K 47/10; A61K 47/24; A61K 47/36; A61K 47/44

(52) U.S. Cl. CPC A61K 9/107; A61K 9/0019; A61K 31/337; A61K 31/704; A61K 45/06; A61K 47/10; A61K 47/24; A61K 47/36; A61K 47/44

(57) ABSTRACT

Disclosed herein is composition for treating a tumor in a subject in need thereof that comprises an emulsion comprising: an aqueous carrier; and liquid lipid phase dispersed into droplets within the aqueous carrier, and a first chemotherapeutic agent within the lipid phase. In certain embodiments, the first chemotherapeutic agent comprises a first plurality of chemotherapeutic agent crystals. In certain alternative embodiments, the first chemotherapeutic agent is dissolved with the lipid phase. In certain implementations, the composition further comprises a second plurality of chemotherapeutic agent crystals within the aqueous carrier, but not the lipid phase and wherein the second plurality of chemotherapeutic agent crystals dissolves and elutes from the emulsion at a faster rate than the first plurality chemotherapeutic agent crystals. In certain implementations, the composition further comprises one or more additional chemotherapeutic agents, different from the first chemotherapeutic agent.

### CROSS-REFERENCE TO RELATED APPLICATION(S)

This application claims priority to U.S. Provisional Application No. 63/454,507, filed Mar. 24, 2023, and entitled “SUSTAINED RELEASE CANCER THERAPEUTICS FORMULATIONS,” which is hereby incorporated by reference in its entirety under 35 U.S.C. § 119(e).

### BACKGROUND

Multifocal tumors have few surgical treatment options in liver, pancreas, renal, and lung cancers. There is a need in the art for compositions and methods that are effective in providing sustained localized delivery of antitumor agents to maximize efficacy and minimize systemic effects. Similarly, there is a need for treatment targeted to a single tumor prior to tumor excision.

### BRIEF SUMMARY

Disclosed herein is composition for treating a tumor in a subject in need thereof that comprises an emulsion comprising: an aqueous carrier; and liquid lipid phase dispersed into droplets within the aqueous carrier, and a first chemotherapeutic agent within the lipid phase. In certain embodiments, the first chemotherapeutic agent comprises a first plurality of chemotherapeutic agent crystals. In certain alternative embodiments, the first chemotherapeutic agent is dissolved with the lipid phase. In certain implementations, the composition further comprises a second plurality of chemotherapeutic agent crystals within the aqueous carrier, but not the lipid phase and wherein the second plurality of chemotherapeutic agent crystals dissolves and elutes from the emulsion at a faster rate than the first plurality chemotherapeutic agent crystals. In certain implementations, the composition further comprises one or more additional chemotherapeutic agents, different from the first chemotherapeutic agent.

According to certain embodiments, the lipid phase is from about 10% to about 40% (w/v) of the composition. In certain embodiments, the lipid phase includes one or more triglycerides. In certain embodiments, the lipid phase comprises soybean oil in an amount from about 15% to 80% (v/v) and one or more medium chain triglycerides in amount from about 15% to about 20% (v/v). certain embodiments, the lipid phase comprises tributyrate and/or stearate. In certain further embodiments, the lipid phase also includes one or more oil and/or wax.

In certain embodiments, the lipid phase further comprises an oil and/or wax that is solid at 25° C. in an amount of from about 5% to about 30% of the lipid phase (w/w) and wherein the oil and/or wax coats the chemotherapeutic agent crystals.

In certain embodiments, the aqueous carrier further comprises an emulsifier and a polyol. In certain implementations, the emulsifier is hyaluronic acid such as, for example, a tyramine substituted hyaluronic acid. In certain embodiments, the polyol is glycerol and is present in an amount of from about 0.25 to about 2.5% (w/v) of the composition.

According to certain embodiments, the lipid phase further comprises a phospholipid. In certain implementations, the phospholipid is present in an amount from about 0.1% to about 2.0% of the lipid phase. According to certain further embodiments, the lipid phase further comprises an antioxidant. In exemplary implementations, the antioxidant is present in an amount of from about 0.01% to about 1% (w/v) of the composition.

According to certain embodiments, the emulsion further comprises lecithin. In certain implementations, the lecithin is present in amount of from 0.1-5% (w/v) of the emulsion.

In certain embodiments, the emulsion further comprises dextrose, and wherein dextrose is present in amount of from about 1-2% (w/v) of the emulsion.

In certain embodiments, the chemotherapeutic agent is present in an amount from about 0.1 mg/g soybean oil to about 300 mg/g soybean oil. In certain implementations, the chemotherapeutic agent is selected from anthracyclines, mTOR inhibitors, VEGF-TKI agents, and immune stimulators. In further implementations, the chemotherapeutic agent is docetaxel or doxorubicin.

Further disclosed herein is a composition for treating a tumor in a subject in need thereof, comprising: an emulsion comprising: an aqueous carrier; and liquid lipid phase dispersed into droplets within the aqueous carrier, and a first chemotherapeutic agent within the lipid phase, wherein the first chemotherapeutic agent comprises a first plurality of chemotherapeutic agent crystals and wherein the liquid lipid phase further comprise one or more oil and/or wax that with a melting point above 25° C. and wherein the one or more oil and/or wax coat the first plurality of chemotherapeutic agent crystals. In certain embodiments, wherein the one or more oil and/or wax are coconut oil and/or carnauba wax. In certain implementations, the coconut oil and/or carnauba wax are present in an amount of from about 3% to about 30% of the lipid phase (w/w).

Further disclosed herein is a composition for treating a tumor in a subject in need thereof, comprising an emulsion comprising: a lipid carrier phase; and an aqueous phase dispersed into droplets within the lipid carrier phase, and a first chemotherapeutic agent within the aqueous phase. In certain embodiments, the first chemotherapeutic agent comprises a first plurality of chemotherapeutic agent crystals. In certain implementations, the first chemotherapeutic agent is dissolved with the aqueous phase. According to certain embodiments, the composition also includes a second plurality of chemotherapeutic agent crystals present within the lipid carrier, but not the aqueous phase, and the second plurality of chemotherapeutic agent crystals dissolves and elutes from the emulsion at a faster rate than the first plurality chemotherapeutic agent crystals. In exemplary implementations, the chemotherapeutic agent is hydrophilic and is dissolved within the aqueous phase. In certain embodiments, the composition further comprises one or more additional chemotherapeutic agents, different from the first chemotherapeutic agent.

According to certain embodiments, the emulsion disclosed herein is stable for a period of about 1 month to about 2 years. In further embodiments, the emulsion is stable for a period of about 6 months to about 12 months. In certain embodiments, the emulsion is reversible.

Further disclosed herein is a method of treating a tumor in a subject in need thereof comprising administering to the subject and effective amount of a composition that comprises an emulsion comprising: an aqueous carrier; and lipid phase dispersed into droplets within the aqueous carrier, and a first chemotherapeutic agent within the lipid phase. In certain embodiments, the chemotherapeutic agent comprises a plurality of chemotherapeutic agent crystals, and wherein the composition further comprises a second plurality of chemotherapeutic agent crystals within the aqueous carrier, but not the lipid phase and wherein the second plurality of chemotherapeutic agent crystals dissolves and elutes from the emulsion at a faster rate than the first plurality chemotherapeutic agent crystals. In certain embodiments, the composition is administered directly to the tumor site by guided needle, laparoscopically or post surgically after removal of the tumor. In certain implementations, the chemotherapeutic agent is eluted from the composition over a period of between about 4 and about 7 days.

According to certain embodiments, the composition further comprises an immune stimulator. In certain implementation, the lipid phase includes one or more fatty acid in an amount sufficient to induce a local inflammatory response in the subject at the site of injection (e.g., proximal to the tumor). In these embodiments, the local inflammatory enhances the subject's antitumor immune response, thus increasing the efficacy of the treatment.

Further disclosed herein is a method of treating a cancer of the lymphatic system in a subject in need thereof by administering to the subject a composition disclosed herein.

While multiple embodiments are disclosed, still other embodiments of the disclosure will become apparent to those skilled in the art from the following detailed description, which shows and describes illustrative embodiments of the disclosed apparatus, systems, and methods. As will be realized, the disclosed apparatus, systems, and methods are capable of modifications in various obvious aspects, all without departing from the spirit and scope of the disclosure. Accordingly, the drawings and detailed description are to be regarded as illustrative in nature and not restrictive.

### BRIEF DESCRIPTION OF THE DRAWINGS

FIG. 1 shows docetaxel blood plasma concentration (ng/mL) over time after intramuscular injection with INSB400 compared to docetaxel standard of care treatment, according to certain embodiments.

FIG. 2 shows doxorubicin blood plasma concentration (ng/ml) over time after intramuscular injection with INSB400 compared to docetaxel standard of care treatment, according to certain embodiments.

FIG. 3A shows a microscope image of Sample 1 at 10× magnification, according to certain implementations.

FIG. 3B shows a microscope image of Sample 1 at 20× magnification, according to certain implementations.

FIG. 4A shows a microscope image of Sample 2 at 10× magnification, according to certain implementations.

FIG. 4B shows a microscope image of Sample 2 at 20× magnification, according to certain implementations.

FIG. 5A shows a microscope image of Sample 3 at 10× magnification, according to certain implementations.

FIG. 5B shows a microscope image of Sample 3 at 20× magnification, according to certain implementations.

FIG. 6A shows a microscope image of Sample 4 at 10× magnification, according to certain implementations.

FIG. 6B shows a microscope image of Sample 4 at 20× magnification, according to certain implementations.

FIG. 7A shows a microscope image of Sample 5 at 10× magnification, according to certain implementations.

FIG. 7B shows a microscope image of Sample 5 at 20× magnification, according to certain implementations.

FIG. 8A shows a microscope image of Sample 6 at 10× magnification, according to certain implementations.

FIG. 8B shows a microscope image of Sample 6 at 20× magnification, according to certain implementations.

FIG. 9A shows a microscope image of Sample 7 at 10× magnification, according to certain implementations.

FIG. 9B shows a microscope image of Sample 7 at 20× magnification, according to certain implementations.

FIG. 10A shows a microscope image of Sample 8 at 10× magnification, according to certain implementations.

FIG. 10B shows a microscope image of Sample 8 at 20× magnification, according to certain implementations.

FIG. 11A shows a microscope image of Sample 9 at 10× magnification, according to certain implementations.

FIG. 11B shows a microscope image of Sample 9 at 20× magnification, according to certain implementations.

FIG. 12A shows a microscope image of Sample 10 at 10× magnification, according to certain implementations.

FIG. 12B shows a microscope image of Sample 10 at 20× magnification, according to certain implementations.

FIG. 13A shows a microscope image of Sample 11 at 10× magnification, according to certain implementations.

FIG. 13B shows a microscope image of Sample 11 at 20× magnification, according to certain implementations.

FIG. 14A shows a microscope image of Sample 12 at 10× magnification, according to certain implementations.

FIG. 14B shows a microscope image of Sample 12 at 20× magnification, according to certain implementations.

FIG. 15A shows a microscope image of Sample 13 at 10× magnification, according to certain implementations.

FIG. 15B shows a microscope image of Sample 13 at 20× magnification, according to certain implementations.

FIG. 16A shows a microscope image of Sample 14 at 10× magnification, according to certain implementations.

FIG. 16B shows a microscope image of Sample 14 at 20× magnification, according to certain implementations.

FIG. 17A shows a microscope image of Sample 15 at 10× magnification, according to certain implementations.

FIG. 17B shows a microscope image of Sample 15 at 20× magnification, according to certain implementations.

FIG. 18A shows a microscope image of Sample 16 at 10× magnification, according to certain implementations.

FIG. 18B shows a microscope image of Sample 16 at 20× magnification, according to certain implementations.

FIG. 19A shows a microscope image of Sample 17 at 10× magnification, according to certain implementations.

FIG. 19B shows a microscope image of Sample 17 at 20× magnification, according to certain implementations.

FIG. 20A shows a microscope image of Sample 18 at 10× magnification, according to certain implementations.

FIG. 20B shows a microscope image of Sample 18 at 20× magnification, according to certain implementations.

FIG. 21A shows a microscope image of Sample 19 at 10× magnification, according to certain implementations.

FIG. 21B shows a microscope image of Sample 19 at 20× magnification, according to certain implementations.

FIG. 22A shows a microscope image of Sample 20 at 10× magnification, according to certain implementations.

FIG. 22B shows a microscope image of Sample 20 at 20× magnification, according to certain implementations.

FIG. 23A shows a microscope image of Sample 21 at 10× magnification, according to certain implementations.

FIG. 23B shows a microscope image of Sample 21 at 20× magnification, according to certain implementations.

FIG. 24A shows a microscope image of Sample 22 at 10× magnification, according to certain implementations.

FIG. 24B shows a microscope image of Sample 22 at 20× magnification, according to certain implementations.

FIG. 25A shows a microscope image of Sample 23 at 10× magnification, according to certain implementations.

FIG. 25B shows a microscope image of Sample 23 at 20× magnification, according to certain implementations.

FIG. 26 shows a microscope image of Sample 24 at 40× magnification, according to certain implementations.

FIG. 27 shows a microscope image of Sample 25 at 40× magnification, according to certain implementations.

### DETAILED DESCRIPTION

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present disclosure, example methods and materials are now described.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by systemic reduction of the compound structure using naming conventions, or by commercially available software, such as CHEMDRAW™ (Perkin Elmer Corporation, U.S.A.).

As used in the specification, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

Unless otherwise indicated, references in the specification to parts by weight of a particular element or component in a composition denotes the weight relationship between the element or component and any other elements or components in the composition or article for which a part by weight is expressed, unless expressly described otherwise. Thus, in a compound containing 2 parts by weight of component X and 5 parts by weight component Y, components X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” means that the subsequently described event or circumstance can or cannot occur, and that the description includes instances where said event or circumstance occurs and instances where it does not.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “cancer” refers to cells having the capacity for autonomous growth. Examples of such cells include cells having an abnormal state or condition characterized by rapidly proliferating cell growth. The term is meant to include cancerous growths, e.g., tumors; oncogenic processes, metastatic tissues, and malignantly transformed cells, tissues, or organs, irrespective of histopathologic type or stage of invasiveness. Also included are malignancies of the various organ systems, such as respiratory, cardiovascular, renal, reproductive, hematological, neurological, hepatic, gastrointestinal, and endocrine systems; as well as adenocarcinomas which include malignancies such as most colon cancers, renal-cell carcinoma, prostate cancer and/or testicular tumors, non-small cell carcinoma of the lung, cancer of the small intestine, and cancer of the esophagus. Cancer that is “naturally arising” includes any cancer that is not experimentally induced by implantation of cancer cells into a subject, and includes, for example, spontaneously arising cancer, cancer caused by exposure of a patient to a carcinogen(s), cancer resulting from insertion of a transgenic oncogene or knockout of a tumor suppressor gene, and cancer caused by infections, e.g., viral infections. The term “carcinoma” is art recognized and refers to malignancies of epithelial or endocrine tissues. In some embodiments, the present methods can be used to treat a subject having an epithelial cancer, e.g., a solid tumor of epithelial origin, e.g., lung, breast, ovarian, prostate, renal, pancreatic, or colon cancer.

As used herein, the term “diagnosed” means having been subjected to a physical examination by a person of skill, for example, a physician, and found to have a condition that can be diagnosed or treated by the compounds, compositions, or methods disclosed herein. For example, “diagnosed with cancer” means having been subjected to a physical examination by a person of skill, for example, a physician, and found to have a condition that can be diagnosed or treated by a compound or composition that can reduce tumor size or slow rate of tumor growth. A subject having cancer, tumor, or at least one cancer or tumor cell, may be identified using methods known in the art. For example, the anatomical position, gross size, and/or cellular composition of cancer cells or a tumor may be determined using contrast-enhanced MRI or CT. Additional methods for identifying cancer cells can include, but are not limited to, ultrasound, bone scan, surgical biopsy, and biological markers (e.g., serum protein levels and gene expression profiles). An imaging solution comprising a cell-sensitizing composition of the present disclosure may be used in combination with MRI or CT, for example, to identify cancer cells.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the phrase “identified to be in need of treatment for a disorder,” or the like, refers to selection of a subject based upon need for treatment of the disorder. For example, a subject can be identified as having a need for treatment of a disorder based upon an earlier diagnosis by a person of skill and thereafter subjected to treatment for the disorder. It is contemplated that the identification can, in one aspect, be performed by a person different from the person making the diagnosis. It is also contemplated, in a further aspect, that the identification can be performed by one who subsequently performed the administration.

The terms “antitumor agent” and “chemotherapeutic agent (CA)” are used interchangeably herein and refer to an agent for the treatment of cancer. Typically, an antitumor agent is a cytotoxic anti-neoplastic drug, which is administered as part of a standardized regimen. Without being bound by theory, antitumor agents act by killing cells that divide rapidly, one of the main properties of most cancer cells. Preferably, the antitumor agent is not indiscriminately cytotoxic, but rather targets proteins that are abnormally expressed in cancer cells and that are essential for their growth. Non-limiting examples of antitumor agents include: angiogenesis inhibitors, such as angiostatin K1-3, DL-α-Difluoromethyl-ornithine, endostatin, fumagillin, genistein, minocycline, staurosporine, and (±)-thalidomide; DNA intercalator/cross-linkers, such as Bleomycin, Carboplatin, Carmustinc, Chlorambucil, Cyclophosphamide, cis-Diammineplatinum(II) dichloride (Cisplatin), Melphalan, Mitoxantrone, and Oxaliplatin; DNA synthesis inhibitors, such as (±)-Amethopterin (Methotrexate), 3-Amino-1,2,4-benzotriazine 1,4-dioxide, Aminopterin, Cytosine β-D-arabinofuranoside, 5-Fluoro-5′-deoxyuridine, 5-Fluorouracil, Ganciclovir, Hydroxyurca, and Mitomycin C; DNA-RNA transcription regulators, such as Actinomycin D, Daunorubicin, Doxorubicin, Homoharringtonine, and Idarubicin; enzyme inhibitors, such as S(+)-Camptothecin, Curcumin, (−)-Deguelin, 5,6-Dichlorobenzimidazole 1-β-D-ribofuranoside, Etoposide, Formestane, Fostriccin, Hispidin, 2-Imino-1-imidazoli-dincacetic acid (Cyclocreatine), Mevinolin, Trichostatin A, Tyrphostin AG 34, and Tyrphostin AG 879; gene regulators, such as 5-Aza-2′-deoxycytidine, 5-Azacytidine, Cholecalciferol (Vitamin D3), 4-Hydroxytamoxifen, Melatonin, Mifepristone, Raloxifene, all trans-Retinal (Vitamin A aldehyde), Retinoic acid, all trans (Vitamin A acid), 9-cis-Retinoic Acid, 13-cis-Retinoic acid, Retinol (Vitamin A), Tamoxifen, and Troglitazone; microtubule inhibitors, such as Colchicine, Dolastatin 15, Nocodazole, Paclitaxel, Podophyllotoxin, Rhizoxin, Vinblastine, Vincristine, Vindesine, and Vinorelbine (Navelbine); and unclassified antitumor agents, such as 17-(Allylamino)-17-demethoxygeldanamycin, 4-Amino-1,8-naphthalimide, Apigenin, Brefeldin A, Cimetidine, Dichloromethylene-diphosphonic acid, Leuprolide (Leuprorelin), Luteinizing Hormone-Releasing Hormone, Pifithrin-α, Rapamycin, Sex hormone-binding globulin, Thapsigargin, and Urinary trypsin inhibitor fragment (Bikunin). The antitumor agent may be a neoantigen. Neoantigens are tumor-associated peptides that serve as active pharmaceutical ingredients of vaccine compositions which stimulate antitumor responses and are described in US 2011-0293637, which is incorporated by reference herein in its entirety. The antitumor agent may be a monoclonal antibody such as rituximab, alemtuzumab, Ipilimumab, Bevacizumab, Cetuximab, panitumumab, and trastuzumab, Vemurafenib imatinib mesylate, erlotinib, gefitinib, Vismodegib, 90Y-ibritumomab tiuxetan, 131I-tositumomab, ado-trastuzumab emtansine, lapatinib, pertuzumab, ado-trastuzumab emtansine, regorafenib, sunitinib, Denosumab, sorafenib, pazopanib, axitinib, dasatinib, nilotinib, bosutinib, ofatumumab, obinutuzumab, ibrutinib, idelalisib, crizotinib, erlotinib (Tarceva®), afatinib dimaleate, ceritinib, Tositumomab and 131I-tositumomab, ibritumomab tiuxetan, brentuximab vedotin, bortezomib, siltuximab, trametinib, dabrafenib, pembrolizumab, carfilzomib, Ramucirumab, Cabozantinib, vandetanib, The antitumor agent may be a cytokine such as interferons (INFs), interleukins (ILs), or hematopoietic growth factors. The antitumor agent may be INF-α, IL-2, Aldesleukin, IL-2, Erythropoietin, Granulocyte-macrophage colony-stimulating factor (GM-CSF) or granulocyte colony-stimulating factor. The antitumor agent may be a targeted therapy such as toremifene, fulvestrant, anastrozole, exemestane, letrozole, ziv-aflibercept, Alitretinoin, temsirolimus, Tretinoin, denileukin diftitox, vorinostat, romidepsin, bexarotene, pralatrexate, lenaliomide, belinostat, pomalidomide, Cabazitaxel, enzalutamide, abiraterone acetate, radium 223 chloride, or everolimus. The antitumor agent may be a checkpoint inhibitor such as an inhibitor of the programmed death-1 (PD-1) pathway, for example an anti-PD1 antibody (Nivolumab). The inhibitor may be an anti-cytotoxic T-lymphocyte-associated antigen (CTLA-4) antibody. The inhibitor may target another member of the CD28 CTLA4 Ig superfamily such as BTLA, LAG3, ICOS, PDL1 or KIR. A checkpoint inhibitor may target a member of the TNFR superfamily such as CD40, OX40, CD137, GITR, CD27 or TIM-3. Additionally, the antitumor agent may be an epigenetic targeted drug such as HDAC inhibitors, kinase inhibitors, DNA methyltransferase inhibitors, histone demethylase inhibitors, or histone methylation inhibitors. The epigenetic drugs may be Azacitidine, Decitabine, Vorinostat, Romidepsin, or Ruxolitinib.

The major categories that some anti-proliferative agents fall into include antimetabolite agents, alkylating agents, antibiotic-type agents, hormonal anticancer agents, immunological agents, interferon-type agents, and a category of miscellaneous antineoplastic agents. Some anti-proliferative agents operate through multiple or unknown mechanisms and can thus be classified into more than one category.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, intradermal administration, buccal administration, and parenteral administration, including injectable such as intravenous (“I.V.”) administration, intra-arterial administration, intramuscular (“I.M”) administration, and subcutaneous administration. Administration can be continuous or intermittent.

The term “contacting” as used herein refers to bringing a disclosed composition and a cell (e.g., a tumor cell), a target receptor, or other biological entity together in such a manner that the compound can affect the activity of the target, either directly; i.e., by interacting with the target itself, or indirectly; i.e., by interacting with another molecule, co-factor, factor, or protein on which the activity of the target is dependent.

As used herein, the terms “effective amount” and “amount effective” refer to an amount that is sufficient to achieve the desired result or to have an effect on an undesired condition. For example, The specific effective amount for any particular subject will depend upon a variety of factors, including the disorder being diagnosed and the severity of the disorder; the specific composition employed; the age, body weight, general health, sex and diet of the patient; the time of administration; the route of administration; the rate of excretion of the specific compound employed; the duration of the diagnosis; drugs used in combination or coincidental with the specific compound employed and like factors well known in the medical arts. For example, it is well within the skill of the art to start doses of a compound at levels lower than those required to achieve the desired diagnostic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. Furthermore, effective dosages may be estimated initially from in vitro assays. For example, an initial dosage for use in animals may be formulated to achieve a circulating blood or serum concentration of active compound that is at or above an IC50 of the particular compound as measured in an in vitro assay. Calculating dosages to achieve such circulating blood or serum concentrations, taking into account the bioavailability of the particular active agent, is well within the capabilities of skilled artisans. For guidance, the reader is referred to Fingl & Woodbury, “General Principles,” In: Goodman and Gilman's The Pharmaceutical Basis of Therapeutics, Chapter 1, pp. 1-46, latest edition, Pergamagon Press, which is hereby incorporated by reference in its entirety, and the references cited therein.

The term “pharmaceutically acceptable” describes a material that is not biologically or otherwise undesirable, i.e., without causing an unacceptable level of undesirable biological effects or interacting in a deleterious manner.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions, which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose.

Compounds described herein can contain one or more double bonds and, thus, potentially give rise to cis/trans (E/Z) isomers, as well as other conformational isomers. Unless stated to the contrary, the disclosure includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present disclosure includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Inglod-Prelog system can be used to assign the (R) or (S) configuration to a chiral carbon.

Compounds described herein can comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the disclosure include isotopes of hydrogen, carbon, nitrogen, oxygen, sulfur, fluorine and chlorine, such as ²H, ³H, ¹³C, ¹⁴C, ¹³N, ¹⁵N, ¹⁸O, ¹⁷O, ³⁵S, ¹⁸F and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this disclosure. Certain isotopically-labeled compounds of the present disclosure, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes may be used for their case of preparation and detectability. Further, substitution with heavier isotopes such as deuterium, i.e., ²H, can afford certain advantages resulting from greater metabolic stability, for example increased in vivo half-life or reduced dosage requirements and, hence, may be preferred in some circumstances. Isotopically labeled compounds of the present disclosure and prodrugs thereof can generally be prepared by carrying out the procedures below, by substituting a readily available isotopically labeled reagent for a non-isotopically labeled reagent.

It is known that chemical substances form solids which are present in different states of order which are termed polymorphic forms or modifications. The different modifications of a polymorphic substance can differ greatly in their physical properties. The compounds according to the disclosure can be present in different polymorphic forms, with it being possible for particular modifications to be metastable. Unless stated to the contrary, the disclosure includes all such possible polymorphic forms.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplemental Volumes (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the disclosure as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules, including the compounds are discussed, specifically contemplated is each and every combination and permutation of the compound and the modifications that are possible unless specifically indicated to the contrary. Thus, if a class of molecules A, B, and Care disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the disclosure. Thus, if there are a variety of additional steps that can be performed it is understood that each of these additional steps can be performed with any specific embodiment or combination of embodiments of the methods of the disclosure.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of particles would either completely lack particles, or so nearly completely lack particles that the effect would be the same as if it completely lacked particles. In other words, a composition that is “substantially free of an ingredient or element may still actually contain such item as long as there is no measurable effect thereof.

As used herein, “drug reservoir” means a phase into which an antitumor agent is dissolved that is dissolved distinct from the carrier phase.

As used herein, the terms “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the disclosure.

Chemotherapeutic molecules and biological compounds are eluted from custom formulated multi-phase stable emulsions to produce tumor cell death and initiate apoptosis in an animal with malignant tumors. Depending on chemotherapeutic ingredient contained within the drug reservoir, other useful treatments and clinical applications other than tumor death may be provided, for example, elimination of a fibroid. In general, a stable emulsion containing solid crystal or dissolved chemotherapeutic agents and/or solid phase drug reservoir are contained within a continuous carrier phase that forms a liquid emulsion that allows delivery via syringe and hypodermic needle, through a catheter or through an applicator. Alternative forms may have liquid, solid, or semisolid drug reservoirs or mixtures of multiple drug reservoir forms within the emulsion.

An emulsion can be considered stable if the immiscible phases of the remain unseparated for a commercially useful period. A commercially useful period is one that allows for sufficient time for all relevant testing, shipping, storage, and use to take place. A commercially useful period, in some embodiments, can be between about 1 week to about 5 years. In further embodiments, a commercially useful period can be between about 1 month to about 2 years. In a preferred embodiment, a commercially useful period can be between about 6 months to about 12 months.

An emulsion can also be reversible. A reversible emulsion is one, whereupon the immiscible phases have separated, can be returned to a stable, unseparated state with mixing gentler than required to initially combine the immiscible phases.

The instantly disclosed drug product is a stable emulsion either with or without solid crystals dispersed throughout that provides excellent sustained release performance for small molecules, like doxorubicin and docetaxel. The product provides prolonged elution with the advantage that little to no residual chemotherapeutic agent (CA) or drug product excipient remaining in the body after 14 days. Another advantage to the drug product is that it uses the body's tissues to absorb and remove the drug product in order to deliver a high local concentration of CA while minimizing high systemic levels. Additionally, animal work has shown that the drug product is removed through the lymph system, which is similar to the path of metastatic cancer cells. This allows the formulation to potentially treat malignant cancer cells that may have metastasized. INSB400 is primarily designed to act as a primary chemotherapeutic treatment for solid tumors.

In certain embodiments, additional components, such as fatty acids, histamines, or coley's toxins (pyrogenic agents), are added to the formulation in order to initiate a localized immune response, which would allow the patient's immune system to target tumor specific antigens and clear all tumor cells from the body. In further embodiments, systemic acting immune system stimulants or agents that enhance immune responses are included in the formulation in a concentration that allows an effective dose to be delivered to the body.

Formulations can be created that produce a locally high and systemically low concentration of CA so as to reduce CA related adverse events, while keeping the local tumor exposure concentration high. Additionally, if desired, the formulation can be created so that the drug product produces a high local concentration of CA and keeps a similar systemic exposure as the standard of care to treat potentially metastasized cells present outside of the primary tumor.

Traditional systemic routes of administration for CAs are subject to varied levels of first pass metabolism by the liver depending on the CA used. For that reason, having a drug product that remains at the peri-tumor site while providing high tumor site exposure to CAs can produce beneficial outcomes for the treated patient.

The unique properties of the instantly disclosed composition allows it to store and retain the CA, (e.g., docetaxel) and limit its elution rates. Solid particles within emulsions usually allow for coalescence of a lipid phase and aqueous phases and destabilizes emulsions. In the instantly disclosed formulations, the solid particles do not destabilize the emulsion and the emulsions retain the solid particles dispersed within and throughout the two liquid phases in a homogenous liquid.

Typically, emulsions are unable to deliver a therapeutically effective dose of CA for several days as they cannot carry a sufficient load of CA. This is because emulsions are limited by the solubility limit of the CA in the emulsion components. Stable emulsions typically do not contain solid particles that may initiate coalescence of the lipid and aqueous phases. A previous iteration of the formulation, INSB200, which contained a non-chemotherapeutic API, was able to hold solid API particles in the emulsion up to 28% by weight relative to the lipid components or 12% relative to the entire drug product without phase separation. In some formulations, the described emulsion can carry up to 15% API relative to entire drug product weight. Many lipid components, such as triglycerides, have low CA solubility at body and ambient temperatures preventing them from dissolving pharmaceutically effective amounts of CAs. INSB400, due to its unique component mixture and manufacturing process, creates a stable emulsion with solid CA crystals. In certain embodiments, crystals are present in both the lipid and aqueous phases.

In certain alternative embodiments, the CAs are at a concentration where no solid crystals are present, and a sustained release profile is also achieved. In formulations where the CA is hydrophilic, such as doxorubicin, the continuous phase can be reversed so that water droplets carry the doxorubicin in a continuous oil phase, or alternatively, a hydrophobic CA, such as docetaxel, can be carried by oil droplets in a continuous aqueous phase. Other drug reservoirs may be used, such as solid fatty acid or triglyceride particles, to enhance the tumor toxic response to the drug product. In other examples, the fatty acids and specific triglycerides can be added to the emulsion formulation as part of the liquid emulsion and not as solid particles. In various implementations, the concentration of CA may be about 0.1% (w/w) to about 20% (w/w) in the overall formulation. In further implementations, the concentration of CA may be about 1% (w/w) to about 10% (w/w) in the overall formulation. In further implementations still, the concentration of CA may be about 2% (w/w) to about 5% (w/w) in the overall formulation. In one specific implementation, the concentration of CA may be about 3.009% (w/w), which is about 29.34 mg/mL, in the overall formulation.

In an example provided, a lipophilic CA molecule, like docetaxel, has very low solubility in water, which creates the first barrier of CA diffusion into surrounding tissue. Without being bound by theory, it is believed that the low solubility of the API in the aqueous phase prevents the docetaxel crystals present in the formulation from dissolving quickly. In order for the docetaxel to be dissolved into the aqueous phase, the docetaxel that is already present in the saturated aqueous phase must diffuse out of the emulsion and into the surrounding tissue. The solubility of docetaxel in the aqueous phase limits the rate at which docetaxel can diffuse out of the emulsion. The oil droplets in the emulsion also have a low solubility for docetaxel and this limits the amount of docetaxel that can diffuse from the lipid droplets into the aqueous continuous phase, which creates a second barrier to diffusion. Once the CA in the aqueous phase has been depleted, the CA in the lipid phase must transfer across the liquid/liquid interface going from a low solubility liquid phase into another low solubility liquid. The third barrier to diffusion is the transition of solid API crystals to a liquid solute phase. The lipid liquid and aqueous liquid phase have low CA solubility, so they both limit the mass transfer of the crystal solid phase to a liquid solute phase. Due to these unique elution characteristics of INSB400, formulations last between 3-7 days and necropsy results show the drug product has been cleared or mostly cleared within 14 days. The following are exemplary implementations of three phase formulations, which include a lipid phase, aqueous phase, and solid crystal phase, according to certain embodiments.

In certain embodiments, a relative increase in quantity of CA crystals dispersed within the lipid phase in a formulation leads to an increase in the stability of that formulation. This increase in stability is unexpected, as a higher loading of suspended material in a suspension or emulsion would be expected to decrease stability, as the suspended material could agglomerate and precipitate more easily.

In another iteration of the formulation, both a hydrophilic agent, such as doxorubicin, and a hydrophobic agent, such as Docetaxel, are included in the same emulsion formulation and act on the tumor in two different mechanisms. In these embodiments, the two CAs will also elute from the emulsion at different rates due to their chemical properties, thus allowing for consecutive and tunable exposure of CA concentrations prior to being cleared and eliminated from the tumor site. Treating a tumor with multiple agents simultaneously has the potential to prevent tumor resistance to CAs and provide a more effective treatment. It is understood that other CAs, like cisplatin, can be added to the emulsion so that three or more CAs can be present in the formulation at the same time.

According to certain implementations, the disclosed stable emulsion comprises an aqueous carrier; and lipid phase dispersed into droplets within the aqueous carrier, and a first plurality chemotherapeutic agent crystals within the lipid phase. In certain implementations, a second plurality of chemotherapeutic agent crystals within the aqueous carrier, but not the lipid phase and wherein the second plurality of chemotherapeutic agent crystals dissolves and elutes from the stable emulsion at a faster rate than the first plurality chemotherapeutic agent crystals.

### Lipid Phase

According to certain embodiments, the lipid phase is dispersed within the aqueous phase and contains CA within the dispersed droplets. In certain embodiments, the CA is present in the lipid phase in the form of CA crystals. In further embodiments, the CA is dissolved within the lipid phase. In certain embodiments, the lipid phase may comprise one or more triglycerides and is a liquid at room temperature.

In certain embodiments, higher melting point vegetable oils or waxes (e.g., coconut oil and/or carnauba wax) are included in the lipid phase. In certain implementations of these embodiments, CA crystals within the lipid phase are coated with the higher melting point oils and/or waxes. In certain implementations of these embodiments, such coating of CA crystals further increases the timeframe over which the CA elutes from the drug product. In exemplary implementations, the higher melting point oils and/or waxes are present at an amount of from about 3 to about 30% of weight of the lipid phase.

### Aqueous Phase

According to certain embodiments, the aqueous phase can be dispersed as droplets within a lipid liquid continuous phase. The CA may be present in the aqueous phase as a solute and also may be present in solid crystal phase. Multiple CAs may be present in the aqueous phase droplets. An additional lipophilic CA may be present in the lipid continuous phase along with hydrophilic CAs in the aqueous droplets. In certain embodiments, the aqueous phase may include additional, excipients, salts, and/or antioxidants, as described further below.

### Triglycerides

Triglycerides are an ester of glycerol and three fatty acids that can be all the same fatty acid or mixtures of different fatty acids. The fatty acids can be saturated (no double carbon bonds in the fatty acid chain) or unsaturated (one or more carbon double bonds in the fatty acid chain length) or mixtures of both saturated and unsaturated fatty acid chains. Triglycerides are the primary component in animal fats and plant derived oils.

Triglycerides have long been used as carriers for pharmaceutical ingredients. Castor oil, soy oil, and peanut oils have all been used as carriers for hydrophobic CAs. These formulations are used when a pharmaceutically effective dose can be dissolved within the carrier volume and the volume is small enough to not cause significant discomfort in the recipient.

Triglycerides have been used as a nutritional supplement intravenously or orally and are one of the main energy storage molecules in mammals. Stable emulsions are used in the pharmaceutical industry, but most do not contain solid CA crystals in the formulations.

TABLE 1
Pure Triglycerides

| | Fatty Acid | | Fatty Acid |
|---|---|---|---|
| Common Name | Component | Structure | Saturation |
| Tripropionin | Propanoic acid | C12H20O6 | C3:0 |
| Tributyrin | Butyric acid | C15H26O6 | C4:0 |
| Trivalerin | Valeric acid | C18H32O6 | C5:0 |
| Tricaproin | Caproic acid | C15H26O6 | C6:0 |
| Triheptanoin | Heptanoic acid | C24H44O6 | C7:0 |
| Tricaprylin | Caprylic acid | C₂₇H₅₀O₆ | C8:0 |
| Tripelarigonin | Pelargonic acid | C30H56O6 | C9:0 |
| Tricaprin | Decanoic acid | C₃₃H₆₂O₆ | C10:0 |
| Triundcylin | Undecanoic acid | C36H68O6 | C11:0 |
| Trilaurin | Lauric acid | C39H74O6 | C12:0 |
| Tritridecanoin | Tridecanoic acid | C42H80O6 | C13:0 |
| Trimyristin | Myristic acid | C45H86O6 | C14:0 |
| Tripentadecanoin | Pentadecanoic acid | C48H92O6 | C15:0 |
| Tripalmitin | Palmitic acid | C51H98O6 | C16:0 |
| Trimargarin | Margaric acid | C54H104O6 | C17:0 |
| Tristearin | Stearic acid | C57H110O6 | C18:0 |
| Triolein | Oleic acid | C57H104O6 | C18:1, cis 9 |
| Trinonadecanoylglycerol | Nonadecanoic acid | C60H116O6 | C19:0 |
| Triarachidin | Arachidic acid | C63H122O6 | C20:0 |
| Triheneicosanoin | Heneicosylic acid | C66H128O6 | C21:0 |
| Trierucin | Erucic Acid | C69H128O6 | C22:cis13, 22:1ω9 |
| Tribehenin | Docosanoic acid | C69H134O6 | C22:0 |
| Tritricosanoin | Tricosanoic acid | C72H140O6 | C23:0 |
| Trilignocerin | Lignoceric acid | C75H146O6 | C24:0 |
| Tripentacosylin | Pentacosylic acid | C78H152O6 | C25:0 |
| Tricerotin | Cerotic acid | C81H158O6 | C26:0 |
| Tricarocerin | Carboceric acid | C84H164O6 | C27:0 |
| Trimontanin | Montanic acid | C87H170O6 | C28:0 |
| Trinonacosylin | Nonacosylic acid | C90H176O6 | C29:0 |
| Trimelissin | Melissic acid | C93H182O6 | C30:0 |
| Trihentriacontylin | Hentriacontylic acid | C96H188O6 | C31:0 |
| Trilacceroin | Lacceroic acid | C99H194O6 | C32:0 |
| Tripsyllin | Psyllic acid | C102H200O6 | C33:0 |
| Trigeddin | Geddic acid | C105H206O6 | C34:0 |
| Tricerplastin | Ceroplastic acid | C108H212O6 | C35:0 |
| Trihexatriacontylin | Hexatriacontylic acid | C111H218O6 | C36:0 |
| Triheptatriacontylin | Heptatriacontylic | C114H224O6 | C37:0 |
| Trioctatriacontylin | Octatriacontylic acid | C117H230O6 | C38:0 |
| Trinonatriacontlyin | Nonatriacontylic acid | C120H236O6 | C39:0 |
| Tritetracontylin | Tetracontylic acid | C122H242O6 | C40:0 |
| Triisopalmitin | Isopalmitic acid | C₅₁H₉₈O₆ | C16:0 |
| Triisostearin | Isostearic acid | C57H110O6 | C18:0 |
| Trilinolein | Linoleic acid | C57H98O6 | C18:2n-6 |
| Triheptylundecanoin | Heptylundecanoic | C57H110O6 | C18:0 |
| Tripalmitolein | Palmitoleic acid | C51H92O6 | C16:1-8 |
| Triricinolein | Ricinoleic acid | C57H104O9 | C18:1-9, 11-OH |

TABLE 2
Mixed Triglycerides

| Oil | Primary Fatty Acid Components | Source |
|---|---|---|
| Soybean | 51% linoleic acid, 7-10% α- | Soybean |
| | linolenic acid, 23% oleic acid, 10% | (Glycine max) |
| | palmitic acid and 4% stearic acid. | |
| Coconut | caprylic acid C -8:0 (8%), capric | Coconut |
| | acid, C-10:0, (7%), lauric acid C- | |
| | 12:0, (49%), myristic acid C- | |
| | 14:0(8%), palmitic acid C-16:0 | |
| | (8%), stearic acid C-18:0 (2%), | |
| | oleic acid C-18:1 (6%) and 2% of | |
| | C-18:2 linoleic acid | |
| Corn | 9-12% palmitic acid, 1-3% stearic | Corn germ |
| | acid, 25-35% oleic acid, 40-60% | |
| | linoleic, and trace amounts of lauric | |
| | acid, myristic acid, behenic acid, | |
| | lignoceric acid, erucic acid, and | |
| | palmitoleic acid. | |
| Cottonseed | 52.89% linoleic acid, 25.39% | Cotton |
| | palmitic acid, 16.35% oleic acids, | |
| | together with small amounts of | |
| | 2.33% stearic acid, 1% myristic | |
| | acid, 0.6% palmitoleic acid as well | |
| | as 0.17% linolenic acid | |
| Olive | Oleic acid is the main fatty acid in | Olive |
| | olive oil and accounts for 55-83% | |
| | of total fatty acid content. Olive oil | |
| | also contains variable amounts of | |
| | linoleic acid (3-21%) and linolenic | |
| | acid (<1%). | |
| Palm | Palm oil contains | Palm |
| | approximately 50% saturated fatty | |
| | acids, with 44% palmitic acid | |
| | (C16:0), 5% stearic acid (C18:0), | |
| | and trace amounts of myristic acid | |
| | (C14:0). The unsaturated fatty acids | |
| | are approximately 40% oleic acid | |
| | (C18:1) and 10% polyunsaturated | |
| | linoleic acid (C18:2) and linolenic | |
| | acid (C18:3) | |
| Peanut | oleic acid (45-53%), linoleic acid | Peanut |
| | (27-32%), and palmitic acid (11- | |
| | 14%). | |
| Rapeseed | 60% oleic acid (C18:1), 4% | Rapeseed |
| | palmitic acid (16:0), and 2% stearic | |
| | acid (18:0) | |
| Safflower | Major fatty acids: 55.1-77.0% | Safflower |
| | linoleic acid, 12.45-35.15% oleic | |
| | acid, 5.7-6.81% palmitic acid, and | |
| | 1.88-2.57% stearic acid. | |
| Sesame | The fatty acid composition in | Sesame |
| | sesame seeds consists of abundant | |
| | unsaturated fatty acids: oleic (35.9- | |
| | 42.3%) and linoleic (41.5-47.9%) | |
| | acids from 80% of total fatty acids; | |
| | less than 20% are saturated fatty | |
| | acids, mainly palmitic (7.9-12%) | |
| | and stearic acids (4.8-6.1%) | |
| Sunflower | Sunflower oil contains | Sunflower |
| | approximately 15% saturated, 85% | |
| | unsaturated fatty acid and | |
| | consisting of 14-43% oleic and 44- | |
| | 75% linoleic acids in its unsaturated | |
| | fatty acid content. | |
| Almond | The major fatty acid in almond oil | Almond |
| | is oleic (62.43% in T7-76.34% in | |
| | T4) followed by linoleic (13.97% in | |
| | T4-29.55% in T3) and palmitic | |
| | (4.97% in T2-7.51% inT3) | |
| Beech | The predominant fatty acids present | Beech (Fagus |
| | in triacylglycerols were linoleic | sylvatica L.) |
| | (18:2) and oleic (18:1) followed by | Seed Oil |
| | palmitic (16:0), gadoleic (20:1), | |
| | linolenic (18:3) and stearic (18:0) | |
| | acids in small quantities. | |
| Brazil nut | 0.6% myristic acid, 13.5% palmitic | brazil nut |
| | acid, 0.33% palmitoleic acid, 0.22% | |
| | margaric acid, 11.77% stearic acid, | |
| | 29.9% oleic acid, 42.80% linoleic | |
| | acid, 0.2%0 alpha-linolenic acid, | |
| | 0.54% arachidic acid, 0.21% | |
| | gondoic acid, 0.12% behenic acid, | |
| | 0.34% erucic acid | |
| Cashew | 0.7% myristic acid, 9.93% palmitic | Cashew nut |
| | acid, 0.36% palmitoleic acid, 0.14% | |
| | margaric acid, 8.70% stearic acid, | |
| | 57.24% oleic acid, 20.80% linoleic | |
| | acid, 0.23% alpha-linolenic acid, | |
| | 0.97% arachidic acid, 0.25% | |
| | gondoic acid, 0.39% behenic acid, | |
| | 0.28% erucic acid. | |
| Hazelnut | Fatty acids in hazelnut oil primarily | Hazelnut |
| | consist of oleic acid (73.6%- | (Corylus |
| | 82.6%), linoleic acid (9.8%- | heterophylla) |
| | 16.6%), palmitic acid (4.1%-6.8%) | |
| | and stearic acid (1.6%-3.7%) | |
| Macadamia | oleic acid (C18:1) (45.23-74.86%), | macadamia nut |
| | followed by palmitoleic | |
| | acid (C16:1) (7.81-33.24%), | |
| | palmitic acid (C16:0) (6.44- | |
| | 12.51%), stearic acid (C18:0) | |
| | (1.14-8.51%), and vaccenic acid | |
| | (trans-C18:1) (1.15-4.65%). | |
| Mongongo | elaeostearic acid (18:3) (25%), | Mungongo tree |
| | linoleic acid (18:2) (37%), oleic | (Schinziophyton |
| | acid (18:1) (15%), palmitic and | rautanenii, |
| | stearic acid (18:0) (8-9%, | Euphorbiaceae) |
| | respectively) | |
| Pecan | 4.28% palmitic acid, 0.9% palmitic | pecan nut |
| | acid, 0.10% margaric acid, 1.80% | |
| | stearic acid, 40.63% oleic acid, | |
| | 50.31% linoleic acid, 0.65% alpha- | |
| | linolenic acid, trace amounts of | |
| | arachidic acid, 1.21% gondoic acid, | |
| | 0.16% behenic acid, 0.25% erucic | |
| | acid | |
| Pine Nut | 6.87% palmitic acid, 0.14% | pine nut |
| | palmitoleic acid, 0.10% margaric | |
| | acid, 4.48% stearic acid, 39.55% | |
| | oleic acid, 45.41% linoleic acid, | |
| | 0.63% alpha-linolenic acid, 1.4% | |
| | arachidic acid, 1.6% gondoic acid, | |
| | 0.33% behenic acid, 0.40% erucic | |
| | acid | |
| Pistachio | 0.9% myristic acid, 7.42% palmitic | pistachio nut |
| | acid, 0.70% palmitoleic acid, 0.86% | |
| | stearic acid, 58.19% oleic acid, | |
| | 30.27% linoleic acid, 0.44% alpha- | |
| | linolenic acid, 0.59% arachidic | |
| | acid, 0.60% gondoic acid, 0.34% | |
| | behenic acid, 9.75% erucic acid | |
| Walnut | linoleic acid (60.42-65.77%), oleic | walnut |
| | acid (13.21-19.94%) and linolenic | |
| | acid (7.61-13%) | |
| Pumpkin Seed | The main fatty acids in pumpkin oil | pumpkin seed |
| | seeds are 14.8% palmitic acid | |
| | (C16:0), 25.8% oleic acid (C18:1), | |
| | and 50.9% linoleic acid (C18:2) | |
| Grapefruit Seed | The main fatty acid components in | grapefruit seed |
| | grapefruit seed oil are palmitic acid | |
| | (27.5-28.9%), Linoleic acid (36.6- | |
| | 39.3%), oleic acid (21.1-25.1%), | |
| | linolenic acid (5.9%), stearic acid | |
| | (2.1-2.9%), and myristic acid (0.8- | |
| | 1.2%) | |
| Lemon | 18.8% palmitic, 0.8% | Citrus limon |
| | heptadecanoic, 3.5% stearic, 30.1% | (lemon) seed oil |
| | oleic, 33.4% linoleic, 13.5% | |
| | linolenic, 0.3% arachidic, 0.3% | |
| | eicosenoic, 0.8% behenic, 0.2% | |
| | lignoceric | |
| Orange | 14-22% palmitic, 2-6% stearic, 26- | Citrus |
| | 35% oleic, 35-45% linoleic, 2-6% | aurantium |
| | linolenic, 0.5% arachidic | dulcis (orange) |
| | | seed oil |
| Watermelon | the main fatty acids in watermelon | watermelon |
| Seed | seed oil are linoleic (59.64%), oleic | seed |
| | (18.7%), palmitic (11.30%), and | |
| | stearic (10.24%) | |
| Acai | palmitic acid 23.0%, palmitoleic | acai pulp |
| | acid 5%, stearic acid 1.3%, oleic | |
| | acid 54.4%, linoleic 16.0% and, | |
| | alpha-linolenic 0.8% | |
| Black cumin | The major fatty acids were linoleic | black cumin |
| Seed | acid (50.2%), oleic acid (19.9%), | (Nigella sativa |
| | margaric acid (10.3%), cis-11,14- | L.) seeds |
| | eicosadienoic acid (7.7%) and | |
| | stearic acid (2.5%) | |
| Blackcurrant | The main fatty acid components of | black currant |
| | black currant seed oil are linoleic | seed |
| | (47.5%), alpha-linolenic (14.5%), | |
| | oleic (13.3%), gamma-linolenic | |
| | (12.6%), palmitic (5.6%), | |
| | stearidonic (2.7%), and stearic | |
| | (1.4%) | |
| Borage | Fatty acid composition of 37.9% | borage seeds |
| | linoleic acid, 24.6% gamma- | (Borago |
| | linolenic acid, 14.8% oleic acid, | officinalis) |
| | 10.2% palmitic acid, 3.3% stearic | |
| | acid, and 0.2% of alpha-linolenic | |
| | and arachidic acids. | |
| Evening | Fatty acid composition of 71.6% | evening |
| Primrose | linoleic acid, 12.6% gamma- | primrose e |
| | linolenic acid, 6.6% oleic acid, | (Oenothera |
| | 5.8% palmitic acid, 2.1% stearic | biennis) |
| | acid, 0.3% arachidic acid, and 0.2% | |
| | alpha-linolenic acid | |
| Flaxseed | The main fatty acid components of | flaxseed |
| | flaxseed oil are alpha-linolenic acid | |
| | (39.9-60.42%), oleic acid (13.44- | |
| | 19.39%), linoleic acid (12.25- | |
| | 17.44%), palmitic acid (4.9-8.0%), | |
| | and stearic acid (2.24-4.59%). | |
| Amaranth | The major fatty acid composition in | Amaranthus |
| | amaranth oil is 12.3-25.9% palmitic | species grain oil |
| | acid, 0.7-4.7% stearic acid, 14.8- | |
| | 38.9% oleic acid, and 33.6-55.9% | |
| | linoleic acid. | |
| Apricot | apricot kernel oil contains 60.01- | apricot kernel |
| | 70.56% oleic acid, 19.74-23.52% | |
| | linoleic acid, 2.35-5.97% palmitic | |
| | acid, 0.8-1.5% stearic acid, and | |
| | 0.2-0.9% palmitoleic acid. | |
| Apple Seed | The main fatty acid components of | apple seed |
| | apple seed oil are linoleic acid | |
| | (50.7-51.4%), oleic acid (37.49- | |
| | 38.55%), palmitic acid (6.51- | |
| | 6.60%), stearic acid (1.75-1.96%) | |
| | and arachidic acid (1.49-1.54%) | |
| Argan | Oleic (43-49%), Linoleic (29- | argan seed |
| | 36$%), Palmitic (11-15%), Stearic | |
| | (4-7%), Palmitoleic (0.3-3%), | |
| | Arachidic (<0.5%), Linolenic | |
| | (<0.2%), Behenic (<0.2%), and | |
| | Myristic (<0.1%) | |
| Avocado | oleic fatty acid (47.2%), followed | avocado seed |
| | by palmitic (23.6%), linoleic | |
| | (13.4%), docosadienoic (8.88%), | |
| | palmitoleic (3.58%), linolenic | |
| | (1.60%), eicosenoic (1.29%), and | |
| | myristic acids (0.33%) | |
| Babssu | The molar percentage for the fatty | babssu oil |
| | acids of babassu oil were caproic | |
| | 0.3, caprylic 7.1, capric 8.3, lauric | |
| | 47.3, myristic 14.5, palmitic 7.1, | |
| | stearic 2.0, arachidic 0.1, oleic 12.2, | |
| | and linoleic acid 1.1 | |
| Ben | The main fatty acid composition of | Moringa |
| | Ben oil is 73.6% oleic acid, 6.25% | oleifera seed |
| | palmitic acid, 6.2% behenic acid, | |
| | 4.97% stearic acid, 3.23% arachidic | |
| | acid, 1.81% gadoleic acid, and | |
| | 1.37% palmitoleic acid | |
| Borneo tallow | Palmitic acid 18.0-22.0%, Stearic | Borneo tallow |
| nut | acid 39-44%, and Oleic acid 38-42, | (tengkawang |
| | % | tallow) is |
| | | derived from |
| | | Shorea |
| | | stenoptera, a |
| | | plant growing in |
| | | the East Indies |
| | | and Malaysia |
| Cape Chestnut | 23.8% palmitic acid, 4.5% stearic | Calodendrum |
| | acid, 33.7% oleic acid, 35.6% | capense seed |
| | linoleic acid, 1.4% linolenic acid, | kernel oil |
| | 1.0% arachidic acid, and trace | |
| | amounts of behenic acid and | |
| | myristic acid. | |
| Carob Pod | Primary fatty acid composition of | carob |
| | carob seed oil is oleic | (Ceratonia |
| | (45.0 ± 0.42%), linoleic | siliqua L.) seed |
| | (32.4 ± 0.30%), palmitic | |
| | (16.6 ± 0.15%), and stearic | |
| | (4.7 ± 0.15%) | |
| Cocoa Butter | 23-30% palmitic acid, 32-37% | cocoa beans |
| | stearic acid, 30-37% oleic acid, 2- | |
| | 4% linoleic acid | |
| Cocklebur | fatty acid composition of cocklebur | cocklebur seeds |
| | seeds is 5.59% palmitic acid, 2.14% | |
| | stearic acid, 20.7% oleic acid, | |
| | 68.6% linoleic acid, 0.29% alpha- | |
| | linolenic acid, and 0.18% gamma- | |
| | linolenic acid. | |
| Cohune | 7.5% caprylic, 6.5% capric, 46.5% | Orbignya |
| | lauric, 16% myristic, 9.5% palmitic, | cohune seed oil |
| | 3% stearic, 10% oleic, and 1% | (Cohune) |
| | linoleic | |
| Date Seed | 0.2-0.8% capric acid, 6.6-35.31% | date seed |
| | lauric acid, 0.4-19.3% myristic acid, | |
| | 9.6-13.1% palmitic acid, 0.8-5.67% | |
| | stearic acid, 0.9-1.66% palmitoleic, | |
| | 31.5-52.5% oleic acid, 0.16 -.65% | |
| | gadoleic acid, 4.4-12.2% linoleic | |
| | acid, and 0.9-1.68% linolenic acid, | |

TABLE 3
Typical Saturated Fatty Acids Present in Triglycerides.

| | | | Lipid |
|---|---|---|---|
| Common Name | Chemical Name | Structural Formula | Numbers |
| Propionic acid | Propanoic acid | CH₃CH₂COOH | C3:0 |
| Butyric acid | Butanoic acid | CH₃(CH₂)₂COOH | C4:0 |
| Valeric acid | Pentanoic acid | CH₃(CH₂)₃COOH | C5:0 |
| Caproic acid | Hexanoic acid | CH₃(CH₂)₄COOH | C6:0 |
| Enanthic acid | Heptanoic acid | CH₃(CH₂)₅COOH | C7:0 |
| Caprylic acid | Octanoic acid | CH₃(CH₂)₆COOH | C8:0 |
| Pelargonic acid | Nonanoic acid | CH₃(CH₂)₇COOH | C9:0 |
| Capric acid | Decanoic acid | CH₃(CH₂)₈COOH | C10:0 |
| Undecylic acid | Undecanoic acid | CH₃(CH₂)₉COOH | C11:0 |
| Lauric acid | Dodecanoic acid | CH₃(CH₂)₁₀COOH | C12:0 |
| Tridecylic acid | Tridecanoic acid | CH₃(CH₂)₁₁COOH | C13:0 |
| Myristic acid | Tetradecanoic acid | CH₃(CH₂)₁₂COOH | C14:0 |
| Pentadecylic acid | Pentadecanoic acid | CH₃(CH₂)₁₃COOH | C15:0 |
| Palmitic acid | Hexadecanoic acid | CH₃(CH₂)₁₄COOH | C16:0 |
| Margaric acid | Heptadecanoic acid | CH₃(CH₂)₁₅COOH | C17:0 |
| Stearic acid | Octadecanoic acid | CH₃(CH₂)₁₆COOH | C18:0 |
| Nonadecylic acid | Nonadecanoic acid | CH₃(CH₂)₁₇COOH | C19:0 |
| Arachidic acid | Eicosanoic acid | CH₃(CH₂)₁₈COOH | C20:0 |
| Heneicosylic acid | Heneicosanoic acid | CH₃(CH₂)₁₉COOH | C21:0 |
| Behenic acid | Docosanoic acid | CH₃(CH₂)₂₀COOH | C22:0 |
| Tricosylic acid | Tricosanoic acid | CH₃(CH₂)₂₁COOH | C23:0 |
| Lignoceric acid | Tetracosanoic acid | CH₃(CH₂)₂₂COOH | C24:0 |
| Pentacosylic acid | Pentacosanoic acid | CH₃(CH₂)₂₃COOH | C25:0 |
| Cerotic acid | Hexacosanoic acid | CH₃(CH₂)₂₄COOH | C26:0 |
| Carboceric acid | Heptacosanoic acid | CH₃(CH₂)₂₅COOH | C27:0 |
| Montanic acid | Octacosanoic acid | CH₃(CH₂)₂₆COOH | C28:0 |
| Nonacosylic acid | Nonacosanoic acid | CH₃(CH₂)₂₇COOH | C29:0 |
| Melissic acid | Triacontanoic acid | CH₃(CH₂)₂₈COOH | C30:0 |
| Hentriacontylic | Hentriacontanoic | CH₃(CH₂)₂₉COOH | C31:0 |
| acid | acid | | |
| Lacceroic acid | Dotriacontanoic acid | CH₃(CH₂)₃₀COOH | C32:0 |
| Psyllic acid | Tritriacontanoic acid | CH₃(CH₂)₃₁COOH | C33:0 |
| Geddic acid | Tetratriacontanoic | CH₃(CH₂)₃₂COOH | C34:0 |
| | acid | | |
| Ceroplastic acid | Pentatriacontanoic | CH₃(CH₂)₃₃COOH | C35:0 |
| | acid | | |
| Hexatriacontylic | Hexatriacontanoic | CH₃(CH₂)₃₄COOH | C36:0 |
| acid | acid | | |
| Heptatriacontylic | Heptatriacontanoic | CH₃(CH₂)₃₅COOH | C37:0 |
| acid | acid | | |
| Octatriacontylic | Octatriacontanoic | CH₃(CH₂)₃₆COOH | C38:0 |
| acid | acid | | |
| Nonatriacontylic | Nonatriacontanoic | CH₃(CH₂)₃₇COOH | C39:0 |
| acid | acid | | |
| Tetracontylic acid | Tetracontanoic acid | CH₃(CH₂)₃₈COOH | C40:0 |

TABLE 4
Typical Monounsaturated Fatty Acids Present in Triglycerides

| | | | Lipid Numbers |
|---|---|---|---|
| | | | C-Atoms:Double |
| Common Name | Chemical Name | Molecular Formula | Bonds |
| Undecylenic | cis-10-undecenoic acid | C₁₀H₁₉COOH | 11:1 |
| Myristoleic | cis-9-tetradecenoic acid | C₁₃H₂₅COOH | 14:1 |
| Palmitoleic | cis-9-hexadecenoic acid | C₁₅H₂₉COOH | 16:1 |
| Palmitelaidic | trans-9-hexadecenoic acid | C₁₅H₂₉COOH | 16:1 |
| Petroselinic | cis-6-octadecenoic acid | C₁₇H₃₃COOH | 18:1 |
| Oleic | cis-9-octadecenoic acid | C₁₇H₃₃COOH | 18:1 |
| Elaidic | trans-9-octadecenoic acid | C₁₇H₃₃COOH | 18:1 |
| Vaccenic | cis-11-octadecenoic acid | C₁₇H₃₃COOH | 18:1 |
| Gondoleic | cis-9-eicosenoic acid | C₁₉H₃₇COOH | 20:1 |
| Gondolic | cis-11-eicosenoic acid | C₁₉H₃₇COOH | 20:1 |
| Cetoleic | cis-11-docosenoic acid | C₂₁H₄₁COOH | 22:1 |
| Erucic | cis-13-docosenoic acid | C₂₁H₄₁COOH | 22:1 |
| Nervonic | cis-15-tetracosaenoic acid | C₂₃H45COOH | 24:1 |

TABLE 5
Omega-3 Fatty Acids.

| | | Lipid |
|---|---|---|
| Common name | Chemical name | Numbers |
| Hexadecatrienoic | all-cis 7,10,13- | 16:3 (n-3) |
| acid (HTA) | hexadecatrienoic acid | |
| Alpha-linolenic acid (ALA) | all-cis-9,12,15- | 18:3 (n-3) |
| | octadecatrienoic acid | |
| Stearidonic acid (SDA) | all-cis-6,9,12,15,- | 18:4 (n-3) |
| | octadecatetraenoic acid | |
| Eicosatrienoic acid (ETE) | all-cis-11,14,17- | 20:3 (n-3) |
| | eicosatrienoic acid | |
| Eicosatetraenoic acid (ETA) | all-cis-8,11,14,17- | 20:4 (n-3) |
| | eicosatetraenoic acid | |
| Eicosapentaenoic acid (EPA, | all-cis-5,8,11,14,17- | 20:5 (n-3) |
| Timnodonic acid) | eicosapentaenoic acid | |
| Heneicosapentaenoic | all-cis-6,9,12,15,18- | 21:5 (n-3) |
| acid (HPA) | heneicosapentaenoic acid | |
| Docosapentaenoic | all-cis-7,10,13,16,19- | 22:5 (n-3) |
| acid (DPA, Clupanodonic | docosapentaenoic acid | |
| acid) | | |
| Docosahexaenoic | all-cis-4,7,10,13,16,19- | 22:6 (n-3) |
| acid (DHA, Cervonic acid) | docosahexaenoic acid | |
| Tetracosapentaenoic acid | all-cis-9,12,15,18,21- | 24:5 (n-3) |
| | tetracosapentaenoic acid | |
| Tetracosahexaenoic | all-cis-6,9,12,15,18,21- | 24:6 (n-3) |
| acid (Nisinic acid) | tetracosahexaenoic acid | |

TABLE 6
Omega-6 Fatty Acids

| | | Lipid |
|---|---|---|
| Common name | Chemical name | Numbers |
| Linoleic acid (LA) | all-cis-9,12-octadecadienoic acid | 18:2 (n-6) |
| Gamma-linolenic acid | all-cis-6,9,12-octadecatrienoic | 18:3 (n-6) |
| (GLA) | acid | |
| Eicosadienoic acid | all-cis-11,14-eicosadienoic acid | 20:2 (n-6) |
| Dihomo-gamma-linolenic | all-cis-8,11,14-eicosatrienoic acid | 20:3 (n-6) |
| acid (DGLA) | | |
| Arachidonic acid (AA) | all-cis-5,8,11,14-eicosatetraenoic | 20:4 (n-6) |
| | acid | |
| Docosadienoic acid | all-cis-13,16-docosadienoic acid | 22:2 (n-6) |
| Adrenic acid (AdA) | all-cis-7,10,13,16- | 22:4 (n-6) |
| | docosatetraenoic acid | |
| Docosapentaenoic acid | all-cis-4,7,10,13,16- | 22:5 (n-6) |
| (Osbond acid) | docosapentaenoic acid | |
| Tetracosatetraenoic acid | all-cis-9,12,15,18- | 24:4 (n-6) |
| | tetracosatetraenoic acid | |
| Tetracosapentaenoic acid | all-cis-6,9,12,15,18- | 24:5 (n-6) |
| | tetracosapentaenoic acid | |

Conjugated fatty acids can also be used alone or in mixtures with other fatty acids to create triglycerides.

TABLE 7
Conjugated Fatty Acids.

| Common name | Chemical name | Lipid Number |
|---|---|---|
| Rumenic acid | 9Z,11E-octadeca-9,11-dienoic acid | 18:2 (n-7) |
| | 10E,12Z-octadeca-10,12-dienoic acid | 18:2 (n-6) |
| α-Calendic acid | 8E,10E,12Z-octadecatrienoic acid | 18:3 (n-6) |
| β-Calendic acid | 8E,10E,12E-octadecatrienoic acid | 18:3 (n-6) |
| Jacaric acid | 8Z,10E,12Z-octadecatrienoic acid | 18:3 (n-6) |
| α-Eleostearic acid | 9Z,11E,13E-octadeca-9,11,13-trienoic acid | 18:3 (n-5) |
| β-Eleostearic acid | 9E,11E,13E-octadeca-9,11,13-trienoic acid | 18:3 (n-5) |
| Catalpic acid | 9Z,11Z,13E-octadeca-9,11,13-trienoic acid | 18:3 (n-5) |
| Punicic acid | 9Z,11E,13Z-octadeca-9,11,13-trienoic acid | 18:3 (n-5) |
| Rumelenic acid | 9E,11Z,15E-octadeca-9,11,15-trienoic acid | 18:3 (n-3) |
| α-Parinaric acid | 9E,11Z,13Z,15E-octadeca-9,11,13,15-tetraenoic acid | 18:4 (n-3) |
| β-Parinaric acid | all trans-octadeca-9,11,13,15-tetraenoic acid | 18:4 (n-3) |
| Bosseopentaenoic acid | 5Z,8Z,10E,12E,14Z-eicosapentaenoic acid | 20:5 (n-6) |

TABLE 8
Common Triglycerides

Tripropionin: (AKA: Glycerol tripropionoate):
Tributyrin: (AKA: Glyceryl tributurate)
Trivelerin: (Glycerol triverlerate)
Tricaproin: (AKA: Trihexanoin, glycerol trihexanoate)
Tripelargonin: (AKA: Trinonanoin, Glyceryl pelargonate)
Triheptanoin: (AKA: Glyceryl triheptanoate)
Tricaprin: (AKA: Trioctanoin, Tricaprilin, Glycerol trioctanoate)
Tricaprin: (AKA: Tridecaoin, Glycerol tridcanoate, Glycerol tricaprate)
Triundecylin: (AKA: Triundecanoin, Glycerol triundecanoate)
Trilaurin: (AKA: Glycerol trilaurate, Glycerol tridodecanoate)
Tritridecanoin: (AKA: glyceryl tridecanoate)
Trimyristin: (AKA: Glycerol trimysristate)
Tritridecanoin: (AKA: 2,3-di(pentadecanoyloxy)propyl pentadecanoate
tripentadecanoin)
Tripalmatin: (AKA: Glycerol tripalmitate) formulations.
Tripalmitolein: (AKA: Glyceryl tripalmitoleate)
Trimargarin: (AKA triheptadecanoin)
Tristearin: (AKA: Glyceryl tristearate)
Triolein: (AKA: Glyceryl trioleate)
Triheptylundecanoin: (AKA: 2,3-bis(2-heptylundecanoyloxy)propyl 2-
heptylundecanoate)
Trinonadecanoylglycerol: (AKA: Trinonadecanoylglycerol)
Triarachidin: (AKA: Glyceryl triarachidate)
Trierucin: (AKA: Glycerol trieucate)
Tribehenin (AKA: Tridocosanoin)
Tritricosanoin: (AKA Glyceryl tritricosanoate)
Tripentacosylin
Tricerotin
Tricarocerin: (AKA: 2,3-Di(heptacosanoyloxy)propyl heptacosanoate)
Trimontanin: (AKA: Propane-1,2,3-triyl trioctacosanoate)
Trimelissin: (AKA: 2,3-Di(triacontanoyloxy)propyl triacontanoate)
Trihentriacontylin:
Trilacceroin: (AKA: 2,3-Di(dotriacontanoyloxy)propyl dotriacontanoate)
Tripsyllin
Trigeddin: (AKA: 2,3-Di(tetratriacontanoyloxy)propyl tetratriacontanoate)
Tricerplastin
Trihexatriacontylin
Triheptatria: contylin
Trioctatriacontylin
Trinonatriacontlyin
Tritetracontylin
Triisopalmitin
Triisostearin
Trilinolein
Triricinolein

As would be understood by those in the art, fatty acids can cause an inflammatory response in the subject. In certain circumstances, such as injection of a formulation containing the inflammation-inducing fatty acid oil, the inflammatory response may be desirable in that it can spur the immune system of the subject into action at the drug product location. This local immune response is more desirable to a systemic immune response, as the risk of immune cells targeting healthy cells is reduced. In the instance where a formulation containing the inflammation-inducing fatty acid oil is injected into a tumor in the subject, the inflammation response can provide synergistic effects in combating the tumor both through APIs in the formulation and through an increase a subject's immune response to the tumor.

In various implementations, the concentration of oils, that is triglycerides, free fatty acids, or both, may be about 15% (w/w) to about 80% (w/w) in the overall formulation. In further implementations, the concentration of oils may be about 20% (w/w) to about 50% (w/w) in the overall formulation. In still further implementations, the concentration of oils may be about 30% (w/w) to about 40% (w/w) in the overall formulation In one specific implementation, the concentration of oils may be about 37.62% (w/w), which is about 366.80 mg/mL, in the overall formulation.

### Excipients

According to certain embodiments, small concentrations of other excipients can be added to supplement or improve stability or act as an antioxidant. Naturally sourced triglycerides will contain unsaturated moieties and can polymerize and oxidize in the presence of oxygen. α-tocopherols can be added to the triglyceride prior to sterile filtration to act as an antioxidant without impacting emulsion stability or elution characteristics. Other hydrophobic antioxidants such as lycopene, retinols, carotenoids, and other tocopherols can be used.

Similarly, hydrophilic antioxidants such as ascorbic acid can be added to protect hydrophilic CAs. In certain embodiments of the formulation, sodium hydroxide is used to maintain a desired pH of ˜8 in order to control elution of the API from the lipid excipients. In further embodiments, other salts or weak acids are used to adjust and maintain the formulation at a desired pH. It is within the knowledge of those skilled in the art to adjust the buffering agents, if present, to ensure a stable pH is maintained over time.

In certain embodiments, glycerol is utilized as an aqueous modifier to produce a stable emulsion. In further implementations, other polyols, including but not limited to sorbitols and manitols, are employed to modify the formulation sufficiently to obtain similar results. In various implementations, the concentration of glycerol may be about 0.05% (w/w) to about 15% (w/w) in the overall formulation. In further implementations, the concentration of glycerol may be about 0.1% (w/w) to about 10% (w/w) in the overall formulation. In further implementations still, the concentration of glycerol may be about 1% (w/w) to about 5% (w/w) in the overall formulation. In one specific implementation, the concentration of glycerol may be about 1.743% (w/w), which is about 17.000 mg/mL, in the overall formulation.

According to certain embodiments, hyaluronic acid, hyaluron, and sodium hyaluronate (collectively referred to herein as “HA”) are considered the same compound for this description and is composed of a long chain polymer containing linear glycosaminoglycan (GAG). HAs can vary in molecular weight (MW) and changes in the MW used will impact formulation viscosity and emulsion stability. In the instantly disclosed formulations, hyaluronic acid is used as an emulsifier and aqueous phase thickener. The higher the concentration, the higher the lipid to aqueous phase ratio that can be achieved and thus a higher total dose of CA available for elution. In various implementations, the concentration of HA may be about 0.001% (w/w) to about 5% (w/w) in the overall formulation. In further implementations, the concentration of HA may be about 0.005% (w/w) to about 2.5% (w/w) in the overall formulation. In further implementations still, the concentration of HA may be about 0.01% (w/w) to about 1% (w/w) in the overall formulation. In one specific implementation, the concentration of HA may be about 0.082% (w/w), which is about 0.8000 mg/mL, in the overall formulation.

According to further embodiments, amphipathic polysaccharides that act as a thickener and have both polar hydroxyl and non-polar methyl ether moieties are used to achieve similar effect to HA. In certain embodiments, polyethylene glycol serves as an emulsifier/thickener. In further embodiments, combinations of the foregoing are used to thicken and stabilize the emulsion.

Lecithin, a mixture of phospholipids, obtained from soy or eggs can be used to produce the same result. In certain embodiments, lecithin obtained from other sources such as sunflower seed or canola seed could be used to generate a similar result. Phospholipids, in this instance, is referring to a class of lipids that contain a hydrophilic phosphate group and two hydrophobic fatty acid groups that are joined by an alcohol residue. One skilled in the art will know that the hydrophilic phosphate “head” group can contain different amino acid chemical moieties. Additionally, one skilled in the art will know that the hydrophobic “tail” groups can contain saturated, monounsaturated, and polyunsaturated fatty acids and vary in chain length from 14 to 18 carbons.

In certain embodiments, phospholipids, in combination with other emulsifiers such as, but not limited to, sorbitan esters, polysorbates, propylene oxide, ethoxylates, copolymers, and macromolecules would produce similar results.

In various implementations, the concentration of lecithin, including similar compounds, may be about 0.1% (w/w) to about 20% (w/w) in the overall formulation. In further implementations, the concentration of lecithin may be about 1% (w/w) to about 10% (w/w) in the overall formulation. In still further implementations, the concentration of lecithin may be about 2% (w/w) to about 5% (w/w) in the overall formulation In one specific implementation, the concentration of lecithin may be about 3.009% (w/w), which is about 29.34 mg/mL, in the overall formulation.

According to certain embodiments, emulsion components and the range of amount of such components are shown in Table 9.

TABLE 9
Emulsion Stability Summary

| Component | Min | Max |
|---|---|---|
| Soybean Oil % (v/v) | 15 | 80 |
| Medium Chain Triglyceride % (v/v) | 15 | 20 |
| HA % | 0 | 3 |
| Aqueous Buffered to ~8.0 pH | No | Yes |
| Tween 80% (w/v) | 0 | 5 |
| Lecithin % (w/v) | 0 | 5 |
| Glycerol % (w/v) | 1.7 | 2.25 |
| PEG % (w/v) | 0 | 0.5 |
| Sorbitol % (w/v) | 0 | 2 |
| Dextrose % (w/v) | 1 | 2 |
| CA Concentration (mg/g_{Soybean oil}) | 0 | 300 |
| NaCl in Aqueous | no | Yes |

### Methods of Formulating Multiphase Stable Emulsion

There are two main methods of manufacturing stable emulsions. The first method utilizes a common solvent, such as acetone (acetone is preferred), other ketone solvents, or mixtures thereof, that can dissolve the triglyceride oil, the lipophilic excipients, and the CA. The second method uses hot triglyceride oils to dissolve an effective amount of CA.

### Solvent Method

For the solvent manufacturing method, the CA and lipophilic excipients are added to a solvent, such as acetone. Once the CA is completely dissolved and a clear solution results, the solution can be sterile filtered through a 0.2 micron or smaller pore size filter media. The resulting solution is then subjected to a crystallization unit operation in which the solvent is removed from the solution resulting in the crystallization of the CA component in the liquid triglyceride phase. The solvent will be completely removed through various methods, for example the solvent can be removed by pulling vacuum on the solvent phase and exhausting or condensing the solvent to remove it from the liquid lipid phase. Heat may be used to increase the vapor pressure of the solvent, making it easier to remove from the solution. Air, nitrogen, or other inert gasses can be used to strip any residual solvent from the liquid lipid phase. Once the solvent is removed from the liquid triglyceride phase, the resulting slurry will contain the CA crystals. An aqueous phase containing all remaining emulsifiers and excipients is now sterile filtered through a 0.2 μm filter media and added to the liquid triglyceride phase and the two phases are well mixed to create a homogenous two-phase liquid that is either exposed to a stator-rotor emulsifier or fed to an inline emulsifier to generate a stable homogenous emulsion containing the CA. The resulting emulsion is transferred to an inventory tank/bag where it can be packaged into a vial, syringe, or other delivery device. One advantage of using the above solvent method is its ability to dissolve temperature sensitive CAs into solution without the need for heating. It also allows formation of high concentration CA formulations that cannot be made with the heat process.

### Heat Method

The heat manufacturing method utilizes an unexpected and nonobvious method of dissolving the CA in the lipid phase, which allows the solution to be sterile filtered before creating the emulsion. Many solvents are able to contain more solute in solution as the solvent is heated but, in most cases, a significant amount of the solute is still present in solution at lower temperature such as room temperature (22° C.). In the example of triglyceride oils, the CA is almost completely insoluble in the oil at room temperature but can be increased when the solution is heated. This allows the CA in solution to be sterile filtered when heated. Another unique aspect of this formulation is that if it is brought into contact with the aqueous components when heated and allowed to cool as the emulsion is generated, the resulting crystals are smaller and a larger portion of CA molecules reside within the oil droplets. Without this hot emulsification, too much of the CA is deposited into the formulation as large crystals and the desired elution profile to the tumor over several days is not achieved.

In certain implementations, the composition further comprises a radiopaque contrast agent.

Further disclosed herein is a method of treating cancer in a subject in need thereof comprising administering to the subject and effective amount of a composition comprising stable multiphase emulsion disclosed herein.

In certain implementations, the immiscible carrier phase is a hydrogel, comprised of substituted hyaluronic acid, such as with tyramine moieties, thereby increasing stability while reducing migration of the anti-cancer therapeutics from the tumor site.

In certain embodiments, the antitumor agent is selected from one or more of: angiogenesis inhibitors, such as angiostatin K1-3, DL-α-Difluoromethyl-ornithine, endostatin, fumagillin, genistein, minocycline, staurosporine, and (±)-thalidomide; DNA intercalator/cross-linkers, such as Bleomycin, Carboplatin, Carmustine, Chlorambucil, Cyclophosphamide, cis-Diammineplatinum(II) dichloride (Cisplatin), Melphalan, Mitoxantrone, and Oxaliplatin; DNA synthesis inhibitors, such as (±)-Amethopterin (Methotrexate), 3-Amino-1,2,4-benzotriazine 1,4-dioxide, Aminopterin, Cytosine β-D-arabinofuranoside, 5-Fluoro-5′-deoxyuridine, 5-Fluorouracil, Ganciclovir, Hydroxyurea, and Mitomycin C; DNA-RNA transcription regulators, such as Actinomycin D, Daunorubicin, Doxorubicin, Homoharringtonine, and Idarubicin; enzyme inhibitors, such as S(+)-Camptothecin, Curcumin, (−)-Deguelin, 5,6-Dichlorobenzimidazole 1-β-D-ribofuranoside, Etoposide, Formestane, Fostriecin, Hispidin, 2-Imino-1-imidazoli-dineacetic acid (Cyclocreatine), Mevinolin, Trichostatin A, Tyrphostin AG 34, and Tyrphostin AG 879; gene regulators, such as 5-Aza-2′-deoxycytidine, 5-Azacytidine, Cholecalciferol (Vitamin D3), 4-Hydroxytamoxifen, Melatonin, Mifepristone, Raloxifene, all trans-Retinal (Vitamin A aldehyde), Retinoic acid, all trans (Vitamin A acid), 9-cis-Retinoic Acid, 13-cis-Retinoic acid, Retinol (Vitamin A), Tamoxifen, and Troglitazone; microtubule inhibitors, such as Colchicine, Dolastatin 15, Nocodazole, Paclitaxel, Podophyllotoxin, Rhizoxin, Vinblastine, Vincristine, Vindesine, and Vinorelbine (Navelbine); and unclassified antitumor agents, such as 17-(Allylamino)-17-demethoxygeldanamycin, 4-Amino-1,8-naphthalimide, Apigenin, Brefeldin A, Cimetidine, Dichloromethylene-diphosphonic acid, Leuprolide (Leuprorelin), Luteinizing Hormone-Releasing Hormone, Pifithrin-α, Rapamycin, Sex hormone-binding globulin, Thapsigargin, and Urinary trypsin inhibitor fragment (Bikunin). The antitumor agent may be a neoantigen. Neoantigens are tumor-associated peptides that serve as active pharmaceutical ingredients of vaccine compositions which stimulate antitumor responses and are described in US 2011-0293637, which is incorporated by reference herein in its entirety. The antitumor agent may be a monoclonal antibody such as rituximab, alemtuzumab, Ipilimumab, Bevacizumab, Cetuximab, panitumumab, and trastuzumab, Vemurafenib imatinib mesylate, erlotinib, gefitinib, Vismodegib, 90Y-ibritumomab tiuxetan, 131I-tositumomab, ado-trastuzumab emtansine, lapatinib, pertuzumab, ado-trastuzumab emtansine, regorafenib, sunitinib, Denosumab, sorafenib, pazopanib, axitinib, dasatinib, nilotinib, bosutinib, ofatumumab, obinutuzumab, ibrutinib, idelalisib, crizotinib, erlotinib (Tarceva®), afatinib dimaleate, ceritinib, Tositumomab and 131I-tositumomab, ibritumomab tiuxetan, brentuximab vedotin, bortezomib, siltuximab, trametinib, dabrafenib, pembrolizumab, carfilzomib, Ramucirumab, Cabozantinib, vandetanib, The antitumor agent may be a cytokine such as interferons (INFs), interleukins (ILs), or hematopoietic growth factors. The antitumor agent may be INF-α, IL-2, Aldesleukin, IL-2, Erythropoietin, Granulocyte-macrophage colony-stimulating factor (GM-CSF) or granulocyte colony-stimulating factor. The antitumor agent may be a targeted therapy such as toremifene, fulvestrant, anastrozole, exemestane, letrozole, ziv-aflibercept, Alitretinoin, temsirolimus, Tretinoin, denileukin diftitox, vorinostat, romidepsin, bexarotene, pralatrexate, lenaliomide, belinostat, pomalidomide, Cabazitaxel, enzalutamide, abiraterone acetate, radium 223 chloride, or everolimus. The antitumor agent may be a checkpoint inhibitor such as an inhibitor of the programmed death-1 (PD-1) pathway, for example an anti-PD1 antibody (Nivolumab). The inhibitor may be an anti-cytotoxic T-lymphocyte-associated antigen (CTLA-4) antibody. The inhibitor may target another member of the CD28 CTLA4 Ig superfamily such as BTLA, LAG3, ICOS, PDL1 or KIR. A checkpoint inhibitor may target a member of the TNFR superfamily such as CD40, OX40, CD137, GITR, CD27 or TIM-3. Additionally, the antitumor agent may be an epigenetic targeted drug such as HDAC inhibitors, kinase inhibitors, DNA methyltransferase inhibitors, histone demethylase inhibitors, or histone methylation inhibitors. The epigenetic drugs may be Azacitidine, Decitabine, Vorinostat, Romidepsin, or Ruxolitinib

Also provided herein are kits of pharmaceutical formulations containing the disclosed compounds or compositions. The kits may be organized to indicate a single formulation or combination of formulations. The composition may be sub-divided to contain appropriate quantities of the compound. The unit dosage can be packaged compositions such as packeted powders, vials, ampoules, prefilled syringes, or sachets containing liquids.

The compound or composition described herein may be a single dose or for continuous or periodic discontinuous administration. For continuous administration, a kit may include the compound in each dosage unit. For periodic discontinuation, the kit may include placebos during periods when the compound is not delivered. When varying concentrations of the composition, the components of the composition, or relative ratios of the compound or other agents within a composition over time is desired, a kit may contain a sequence of dosage units.

The kit may contain packaging or a container with the compound formulated for the desired delivery route. The kit may also contain dosing instructions, an insert regarding the compound, instructions for monitoring circulating levels of the compound, or combinations thereof. Materials for performing using the compound may further be included and include, without limitation, reagents, well plates, containers, markers or labels, and the like. Such kits are packaged in a manner suitable for treatment of a desired indication. Other suitable components to include in such kits will be readily apparent to one of skill in the art, taking into consideration the desired indication and the delivery route. The kits also may include, or be packaged with, instruments for assisting with the injection/administration or placement of the compound within the body of the subject. Such instruments include, without limitation, syringe, pipette, forceps, measuring spoon, eye dropper or any such medically approved delivery means. Other instrumentation may include a device that permits reading or monitoring reactions in vitro.

The compound or composition of these kits also may be provided in dried, lyophilized, or liquid forms. When reagents or components are provided as a dried form, reconstitution generally is by the addition of a solvent. The solvent may be provided in another packaging means and may be selected by one skilled in the art.

A number of packages or kits are known to those skilled in the art for dispensing pharmaceutical agents. In one embodiment, the package is a labeled blister package, dial dispenser package, or bottle.

According to certain embodiments, a formulation of anthracycline (doxorubicin) is delivered to a tumor or the space around a tumor via guided needle, laparoscopically or instilled post surgically after removal of the tumor. 100-120 mg of doxorubicin hydrochloride is supplied in aqueous phase droplets dispersed with a continuous lipid phase, contained within a lipid carrier.

mTOR

In certain embodiments, the disclosed stable emulsion is effective in treating tumors such as renal tumors. In these embodiments, the disclosed stable emulsion contains up to 20 mg of mTOR inhibitor such as Rapamycin (Sirolimus). The lipid phase may contain rapamycin in concentrations from 2-20 mg, or higher depending on the lipid component mixture and elution rate. The lipid phase may be present in volume/volume concentrations between 5% and 50%. One trained in the art can develop similar formulations for mTOR inhibitors such as Everolimus, Temsirolimus and similar agents.

### VEGF-TKI

Vascular endothelial growth factor (VEGF) and Tyrosine kinase inhibitors (TKI) such as Sorafenib can be presented in the lipid phase and carried in a hydrogel aqueous formulation and delivered via ultrasound guided needle, fluoroscopy, laparoscopic or visually instilled during surgery to a tumor or peritumor site to deliver a sustained dose of Sorafenib for several days or longer. In one formulation 600-800 mg Sorafenib is contained within lipid phase in an aqueous carrier phase and injected into a solid tumor, injected into the space around the tumor or instilled into a surgical site post tumor excision. One trained in the art can develop similar formulations for VEGF-TKI agents such as lenvatinib, axintinib and similar agents.

### Targeted Therapy

Many biologics such as Bevacizumab and interferon have good water solubility and the formulation can be modified to make a hydrogel component the drug reservoir carried within either a water/saline carrier or within a liquid lipid formulation. The aqueous nanoparticles may be present in volume/volume concentrations between 5% and 50%. One trained in the art can develop similar formulations specific to targeted therapy agents.

Direct Injection into Tumor

In certain embodiments, the therapeutic formulations can be delivered directly inside a solid tumor via guided needle.

### Intravascular Delivery

In certain embodiments the disclosed emulsion is delivered directly to the vasculature and allowed to circulate in the bloodstream for several days until the particles can carrier are absorbed, and the treatment agents delivered systemically to the body. in this embodiment the treatment agent is delivered in a sustained release concentration to ensure a steady delivery of treatment agent for 1-5 days or longer depending on the agent.

Various aspects and embodiments of the present disclosure are defined by the following numbered clauses:
1. A composition for treating a tumor in a subject in need thereof, comprising:
an emulsion comprising:
an aqueous carrier; and liquid lipid phase dispersed into droplets within the aqueous carrier, and a first chemotherapeutic agent within the lipid phase.
2. The composition of clause 1, wherein the first chemotherapeutic agent comprises a first plurality of chemotherapeutic agent crystals.
3. The composition of clause 1, wherein the first chemotherapeutic agent is dissolved with the lipid phase.
4. The composition of clause 2, further comprising a second plurality of chemotherapeutic agent crystals within the aqueous carrier, but not the lipid phase and wherein the second plurality of chemotherapeutic agent crystals dissolves and elutes from the emulsion at a faster rate than the first plurality chemotherapeutic agent crystals.
5. The composition of any of clauses 1-4, wherein the composition further comprises one or more additional chemotherapeutic agents, different from the first chemotherapeutic agent.
6. The composition of any of clauses 1-5, wherein the lipid phase comprises a triglyceride.
7. The composition of any of clauses 1-6, wherein the aqueous carrier further comprises an emulsifier and a polyol.
8. The composition of clause 7, wherein the emulsifier is hyaluronic acid.
9. The composition of clause 7, wherein the polyol is glycerol and is present in an amount of from about 0.25 to about 2.5% (w/v) of the composition.
10. The composition of clause 8, wherein the hyaluronic acid is tyramine substituted hyaluronic acid.
11. The composition of any of clauses 1-10, wherein the lipid phase further comprises a phospholipid present in an amount from about 0.1% to about 2.0% of the lipid phase.
12. The composition of any of clauses 1-11, wherein the lipid phase further comprises an antioxidant present in an amount of from about 0.01% to about 1% (w/v) of the composition.
13. The composition of any of clauses 1-12, wherein the lipid phase is from about 10% to about 40% (w/v).
14. The composition of any of clauses 1-13, wherein the emulsion further comprises lecithin, and wherein lecithin is present in amount of from 0.1-5% (w/v) of the emulsion.
15. The composition of clause 1, wherein the emulsion further comprises dextrose, and wherein dextrose is present in amount of from about 1-2% (w/v) of the emulsion.
16. The composition of clause 1, wherein the emulsion further comprises Tween 80, and wherein Tween 80 is present in amount of from about 0-5% (w/v) of the emulsion.
17. The composition of clause 1, wherein the emulsion further comprises sorbitol, and wherein sorbitol is present in amount of from about 0.1-2% (w/v) of the emulsion.
18. The composition of clause 1, wherein the emulsion further comprises PEG, and wherein PEG is present in amount of from about 0.1-0.5% (w/v) of the emulsion.
19. The composition of clause 1, wherein the lipid phase comprises soybean oil in an amount from about 15% to 80% (v/v) and one or more medium chain triglycerides in amount from about 15% to about 20% (v/v).
20. The composition of clause 19, wherein the chemotherapeutic agent is present in an amount from about 0.1 mg/g soybean oil to about 300 mg/g soybean oil.
21. The composition of clause 1, wherein the chemotherapeutic agent is selected from anthracyclines, mTOR inhibitors, VEGF-TKI agents, and immune stimulators.
22. The composition of any of clauses 1-21, wherein the lipid phase comprises tributyrate and/or stearate.
23. The composition of clause 2, wherein the lipid phase further comprises an oil and/or wax that is solid at 25° C. in an amount of from about 5% to about 30% of the lipid phase (w/w) and wherein the oil and/or wax coats the chemotherapeutic agent crystals.
24. The composition of any of clauses 1-23, wherein the chemotherapeutic agent is docetaxel
25. The composition of any of clauses 1-23, wherein the antitumor agent is doxorubicin.
26. A composition for treating a tumor in a subject in need thereof, comprising:
an emulsion comprising:
a lipid carrier phase; and an aqueous phase dispersed into droplets within the lipid carrier phase, and a first chemotherapeutic agent within the aqueous phase.
27. The composition of clause 26, wherein the first chemotherapeutic agent comprises a first plurality of chemotherapeutic agent crystals.
28. The composition of clause 26, wherein the first chemotherapeutic agent is dissolved with the aqueous phase.
29. The composition of clause 27, further comprising a second plurality of chemotherapeutic agent crystals present within the lipid carrier, but not the aqueous phase, and wherein the second plurality of chemotherapeutic agent crystals dissolves and elutes from the emulsion at a faster rate than the first plurality chemotherapeutic agent crystals.
30. The composition of clause 29, wherein the chemotherapeutic agent is hydrophilic and is dissolved within the aqueous phase.
31. The composition of clause 26, wherein the composition further comprises one or more additional chemotherapeutic agents, different from the first chemotherapeutic agent.
32. A composition for treating a tumor in a subject in need thereof, comprising:
an emulsion comprising:
an aqueous carrier; and liquid lipid phase dispersed into droplets within the aqueous carrier, and a first chemotherapeutic agent within the lipid phase, wherein the first chemotherapeutic agent comprises a first plurality of chemotherapeutic agent crystals and wherein the liquid lipid phase further comprise one or more oil and/or wax that with a melting point above 25° C. and wherein the one or more oil and/or wax coat the first plurality of chemotherapeutic agent crystals.
33. The composition of clause 32, wherein the one or more oil and/or wax are coconut oil and/or carnauba wax.
34. The composition of clause 33, wherein the coconut oil and/or carnauba wax are present in an amount of from about 3% to about 30% of the lipid phase (w/w).
35. The composition of any of clauses 1-34, wherein the emulsion is stable for a period of about 1 month to about 2 years.
36. The composition of clause 35, wherein the emulsion is stable for a period of about 6 months to about 12 months.
37. The composition of any of clauses 1-36, wherein the emulsion is reversible.
38. A method of treating a tumor in a subject in need thereof comprising administering to the subject and effective amount of a composition comprising:
An emulsion comprising:
an aqueous carrier; and lipid phase dispersed into droplets within the aqueous carrier, and a first chemotherapeutic agent within the lipid phase.
39. The method of clause 38, wherein the chemotherapeutic agent comprises a plurality of chemotherapeutic agent crystals, and wherein the composition further comprises a second plurality of chemotherapeutic agent crystals within the aqueous carrier, but not the lipid phase and wherein the second plurality of chemotherapeutic agent crystals dissolves and elutes from the emulsion at a faster rate than the first plurality chemotherapeutic agent crystals.
40. The method of clauses 38 or 39, wherein the composition is administered directly to the tumor site by guided needle, laparoscopically or post surgically after removal of the tumor.
41. The method of any of clauses 38-40, wherein the chemotherapeutic agent is eluted from the composition over a period of between about 4 and about 7 days.
42. The method of any of clauses 38-41, wherein the composition further comprises an immune stimulator.
43. The method of any of clauses 38-42, wherein the lipid phase includes one or more fatty acid in an amount sufficient to induce a local inflammatory response in the subject at the site of injection.
44. A method of treating a cancer of the lymphatic system in a subject in need thereof, comprising: administering to the subject a composition of any of clauses 1-37.

### EXPERIMENTAL

The following examples are put forth so as to provide those of ordinary skill in the art with a complete disclosure and description of how the compounds, compositions, articles, devices and/or methods claimed herein are made and evaluated and are intended to be purely exemplary of the disclosure and are not intended to limit the scope of what the inventors regard as their disclosure. However, those of skill in the art should, in light of the present disclosure, appreciate that many changes can be made in the specific embodiments which are disclosed and still obtain a like or similar result without departing from the spirit and scope of the disclosure.

### Example 1-Preparation of Stable Emulsion

Stable emulsions containing the CA docetaxel or doxorubicin were prepared according to the following protocol:
1. Docetaxel or doxorubicin was added to soy oil. (up to 13% by weight of oil).
2. 0.75 g of lecithin was added to the soy oil mixture.
3. CA/Oil slurry was heated to 100 C with mixing the slurry vigorously until the crystals of CA are completely dissolved into solution.
4. Soybean oil containing the CA was sterile filtered using 0.2 μm vacuum filter at temperature (100 C).
5. An aqueous solution of 0.15% sodium and 0.51% g glycerol (w/v) was prepared at 4° C.
6. The aqueous solution was heated to 100° C. and sterile filtered by passing it through a 0.2 μm filter media before combining it with the hot soy oil, lecithin, and CA base solution.
7. The two phases in the main mixing vessel were emulsified with a rotor-stator emulsifier or by feeding the two-phase mixture to an in line emulsifier until a stable emulsion formed.
8. Emulsification was continued until mixture cooled to room temperature for subsequent use or storage.

### Example 2-Docetaxel Plasma Concentration in Rats

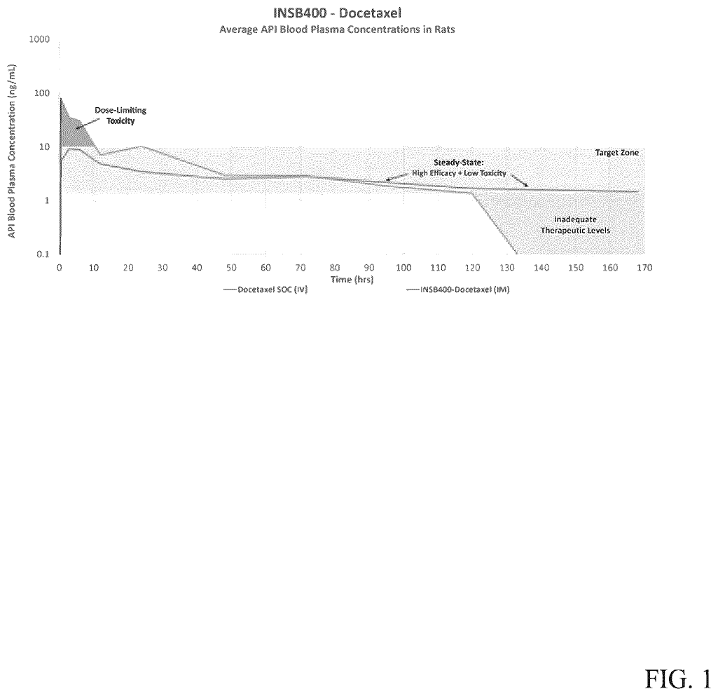

Rats received I.M. injections of a docetaxel stable emulsion prepared according to the protocol in Example 1 or a standard of care docetaxel delivered via I.V. injection. FIG. 1 shows the docetaxel blood plasma concentration over time for a 9.6 mg/Kg stable emulsion formulation. The docetaxel positive control line (6.45 mg/kg) shows the standard of care response. The stable emulsion formulation provides a significant amount of docetaxel for greater than 170 hours and significantly exceeded the SOC docetaxel treatment group.

### Example 3-Doxorubicin Plasma Concentration in Rats

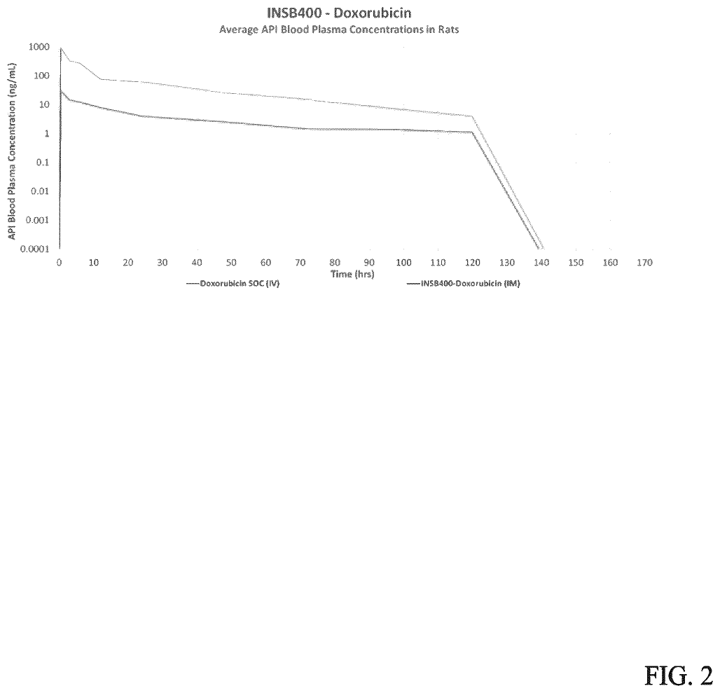

Rats received I.M. injections of a doxorubicin stable emulsion prepared according to the protocol in Example 1 or a standard of care doxorubicin delivered via I.V. injection. FIG. 2 shows the doxorubicin blood plasma concentration over time for a 6.48 mg/kg stable emulsion formulation. The docetaxel positive control line shows standard of care response. The stable emulsion formulation provides a significant amount of docetaxel for greater than 120 hours and reduces systemic exposure to the CA.

### Formula Variations

In Examples 4-10, a base formula is used with various alternations made for each example. The base formulation is given below in Table 10. Mixing of components in the base formula is done using a benchtop mixer with a standard 2-inch head at 6000 RPM. This base formulation, and the various alternations in these Examples, are merely illustrative implementations and are not intended to be restrictive.

TABLE 10
Base Formulation

| Material Name | % (w/w) | mg/mL |
|---|---|---|
| API | 3.009 | 29.34 |
| Soybean Oil | 37.62 | 366.8 |
| Glycerin, Natural | 1.743 | 17.000 |
| Lipoid S75 (Lecithin) | 2.564 | 25.000 |
| Sodium Hyaluronate | .082 | 0.8000 |
| Plant Generated Water for injection (WFI) | 54.94 | 561.2 |

### Example 4-Types of Lecithin

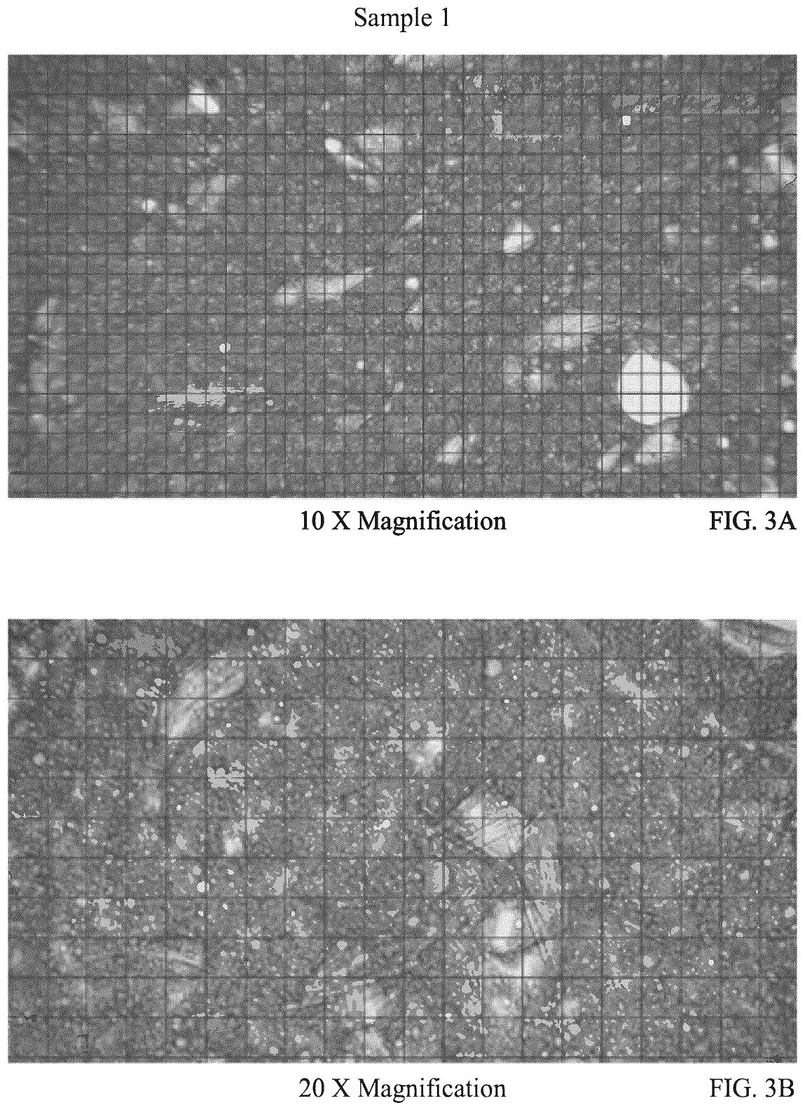

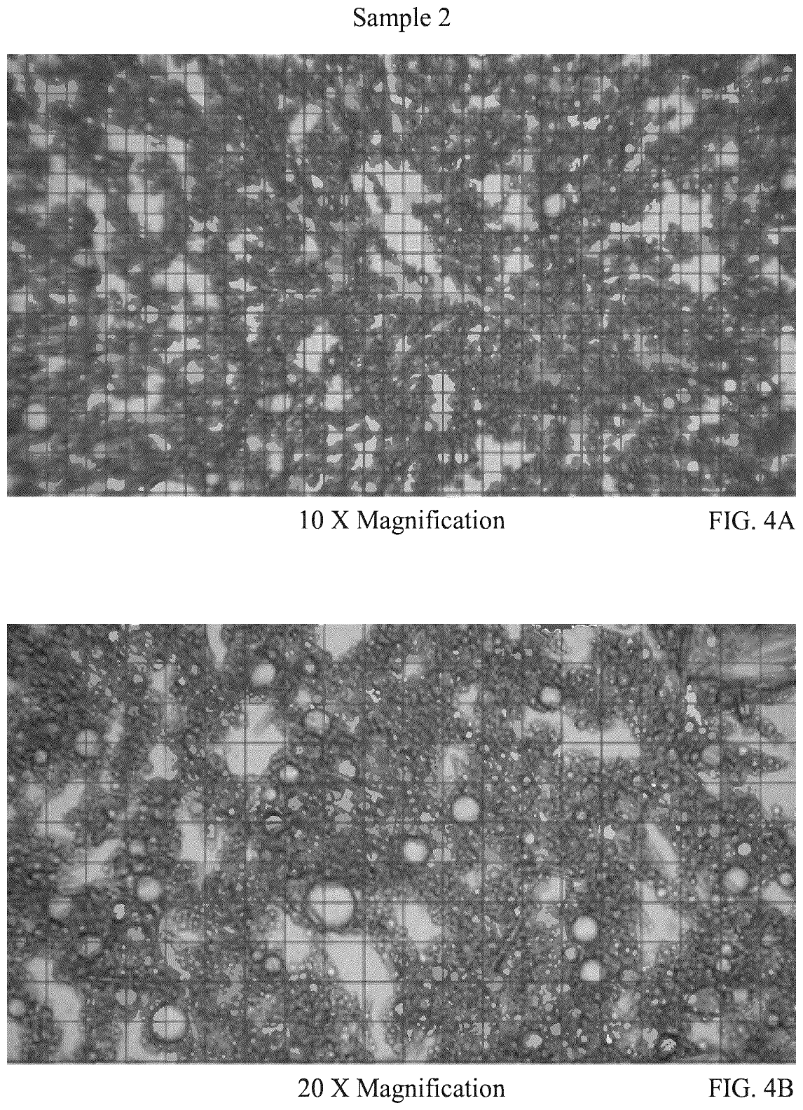

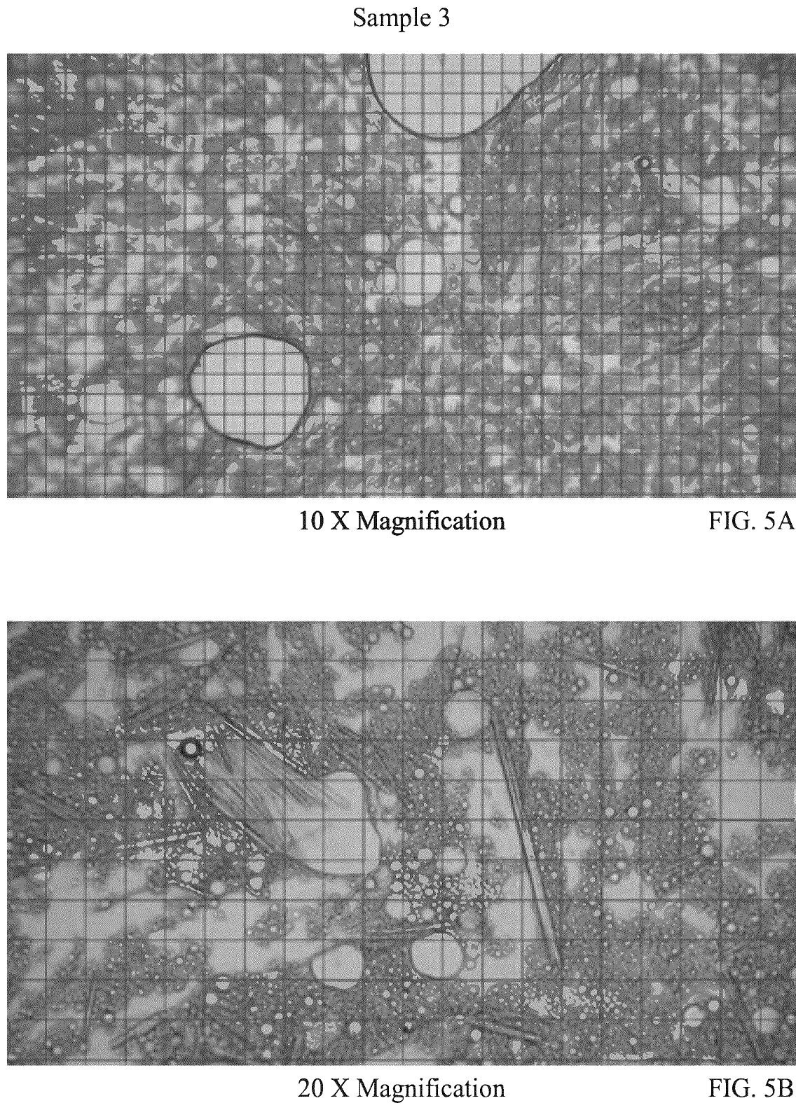

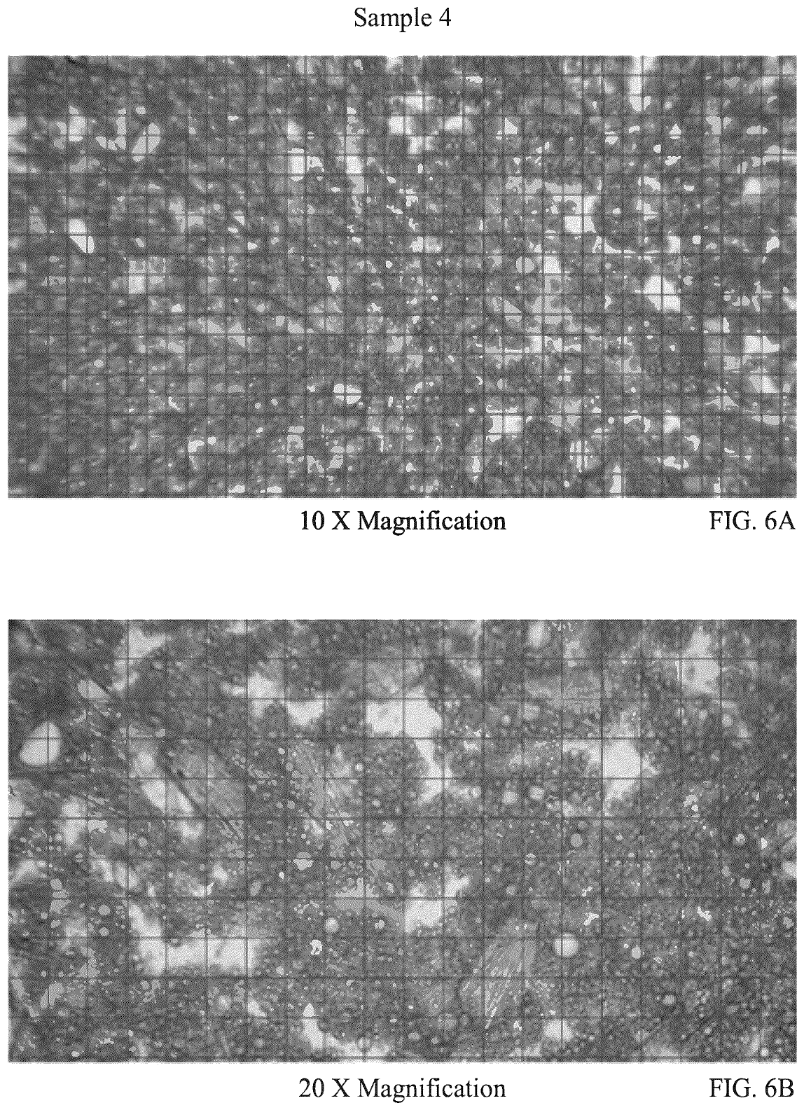

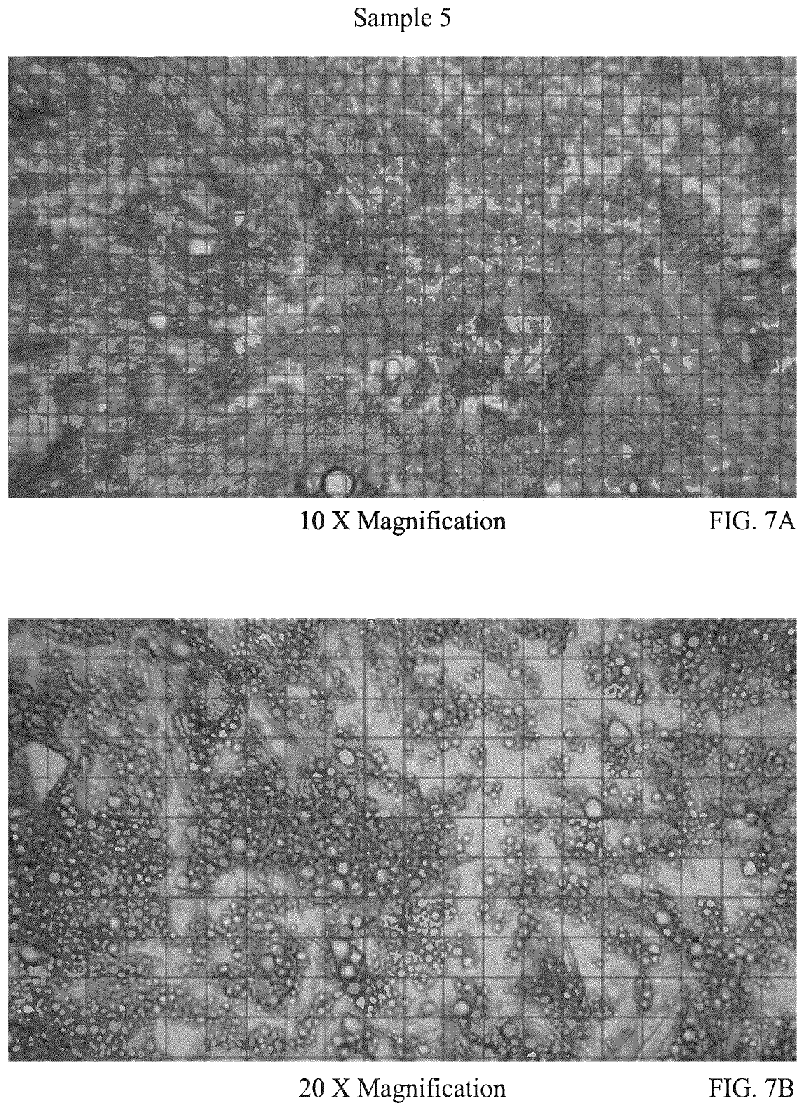

As would be understood, lecithin composition changes depending on where it is sourced (plant, animal, egg), whether it is de-oiled, and degree of processing. Lecithin with high percentages of phosphatidylcholine is often used for injectable products and must meet the USP National Formulary requirements for injection including a phosphatidylcholine concentration >70%. Lecithin with lower concentrations of phosphatidylcholine is typically used for oral and topical drug products, food, and cosmetics. The lecithin with lower phosphatidylcholine results in more stable emulsions with lower energy input during formation, but higher phosphatidylcholine concentration Lecithin still form stable emulsions but require higher energy input during formation. In this Example, five samples, samples 1-5, were developed and analyzed using lecithin of differing composition.
Sample 1 uses the base formulation but with Spectrum NF (50-60% phosphatidylcholine) replacing the lecithin component. Images of the resulting material are shown under 10× magnification in FIG. 3A and under 20× magnification in FIG. 3B. The oil droplets are small, and the API crystals are long needle form. The emulsion remained stable for 14 days. The sample forms a white opaque emulsion/suspension.
Sample 2 uses the base formulation but with Cargill Metarin Lecithin (19-27% phosphatidylcholine) replacing the lecithin component. Images of the resulting material are shown under 10× magnification in FIG. 4A and under 20× magnification in FIG. 4B. The oil droplets are small, and the API crystals are long needle form. The emulsion remained stable for 1 day. The sample forms a white opaque emulsion/suspension.
Sample 3 uses the base formulation but with Cargill Epikuron Lecithin (19-27% phosphatidylcholine) replacing the lecithin component. Images of the resulting material are shown under 10× magnification in FIG. 5A and under 20× magnification in FIG. 5B. The oil droplets are small, and the API crystals are long needle form. The emulsion remained stable for 1 day but can be re-suspended or re-emulsified into an acceptable emulsion through agitation, such as shaking. The sample forms a white opaque emulsion/suspension.
Sample 4 uses the base formulation but with Lipoid S75 (>70% phosphatidylcholine) replacing the lecithin component. Images of the resulting material are shown under 10× magnification in FIG. 6A and under 20× magnification in FIG. 6B. The oil droplets are small, and the API crystals are long needle form. The emulsion remained stable for 4 days but can be re-suspended or re-emulsified into an acceptable emulsion through agitation, such as shaking. The sample forms a white opaque emulsion/suspension.
Sample 5 uses the base formulation but with Lipoid S80 (73-79% phosphatidylcholine) replacing the lecithin component. Images of the resulting material are shown under 10× magnification in FIG. 7A and under 20× magnification in FIG. 7B. The oil droplets are small, and the API crystals are long needle form. The emulsion remained stable for 1 day but can be re-suspended or re-emulsified into an acceptable emulsion through agitation, such as shaking. The sample forms a white opaque emulsion/suspension.

### Example 6 Lecithin Concentration Relative to Soybean Oil

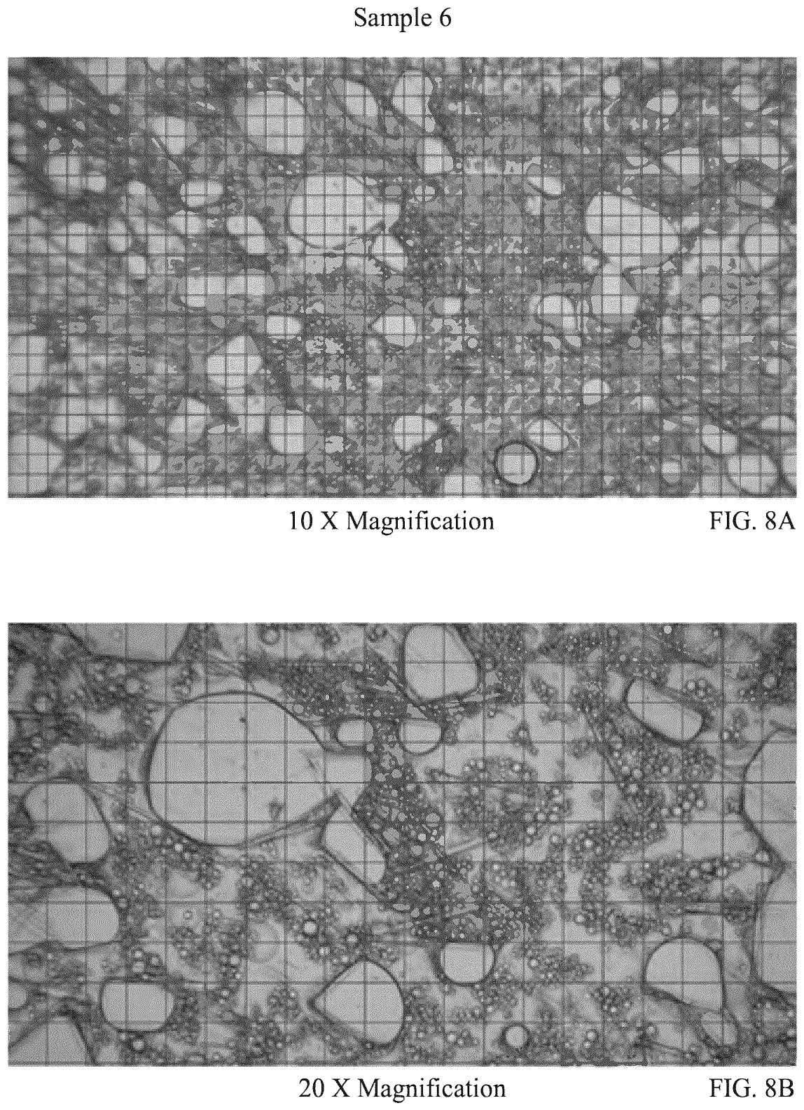

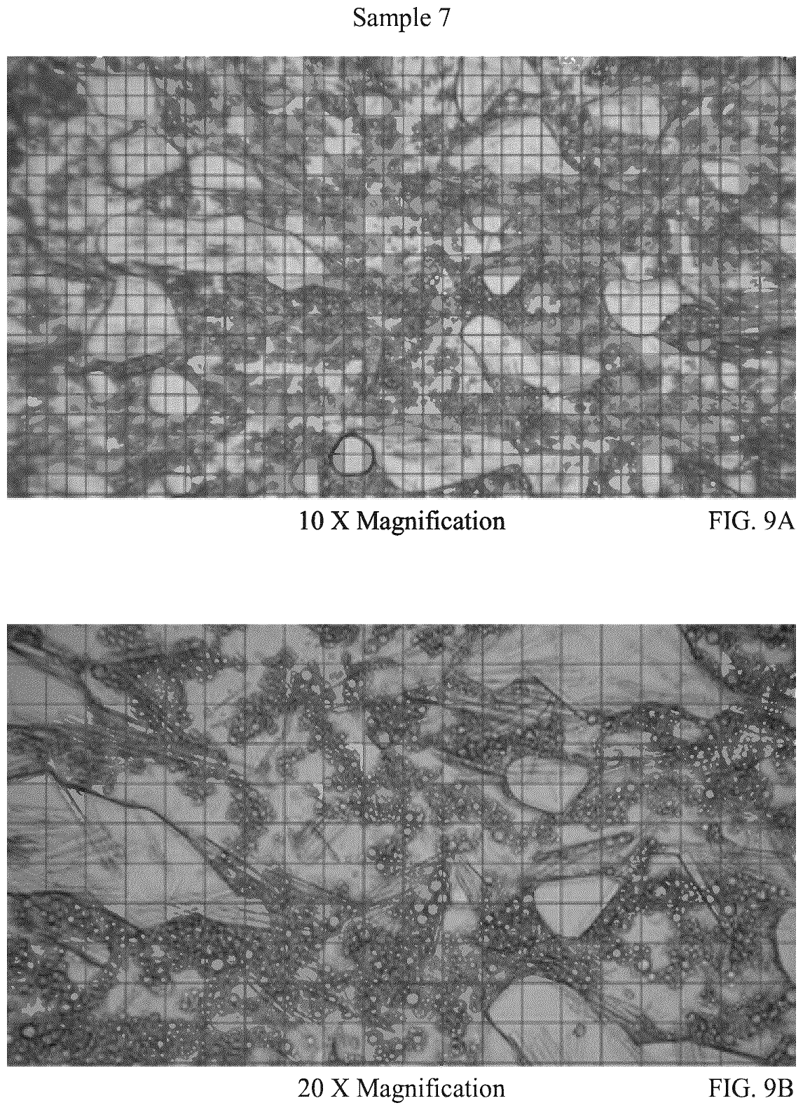

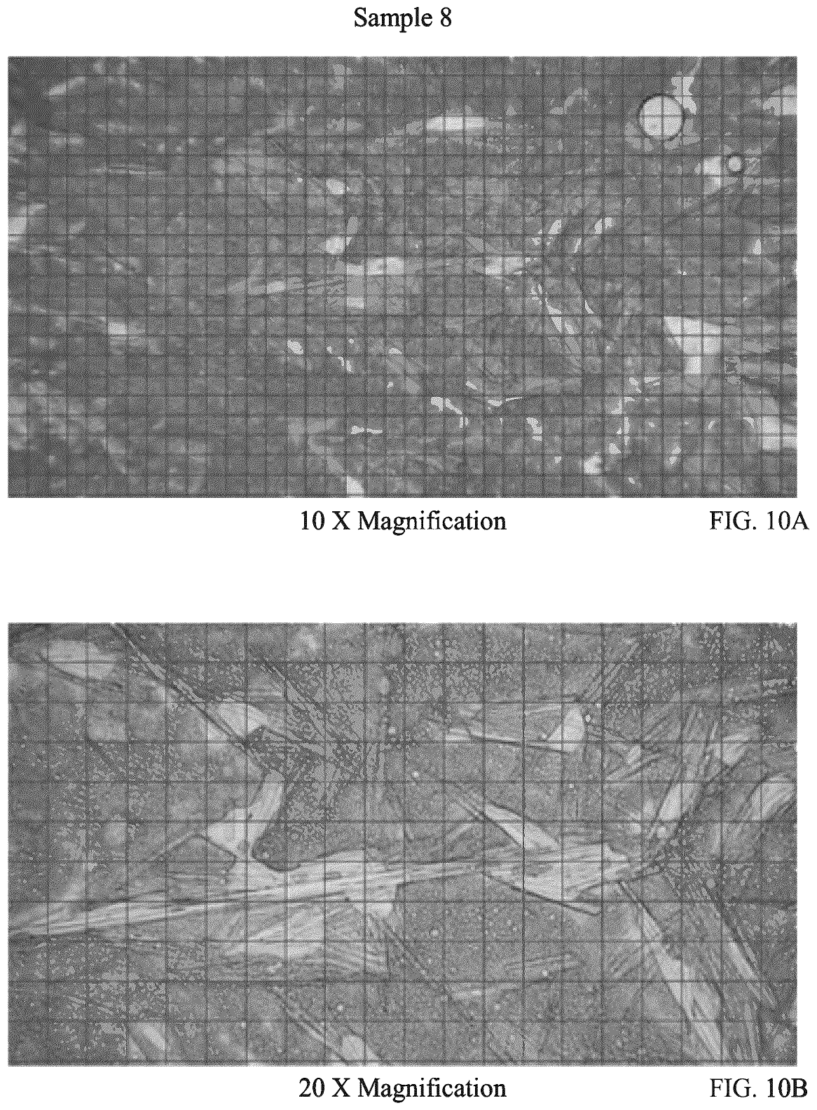

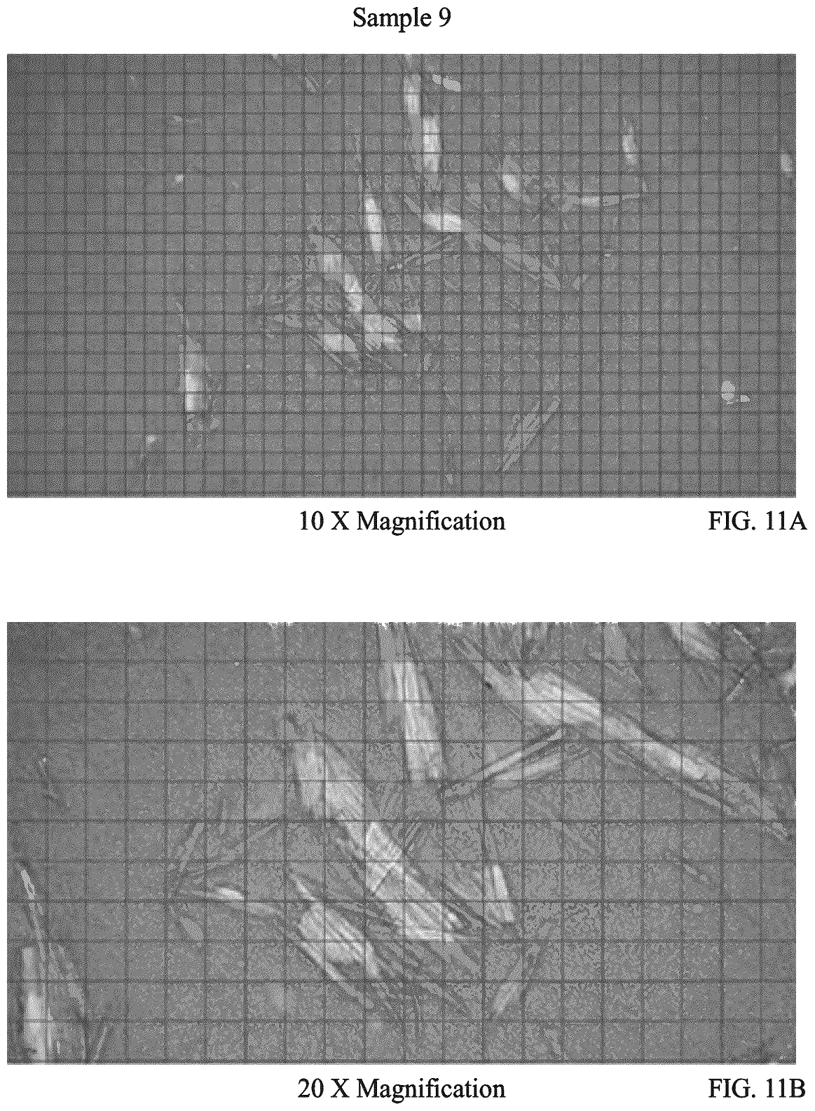

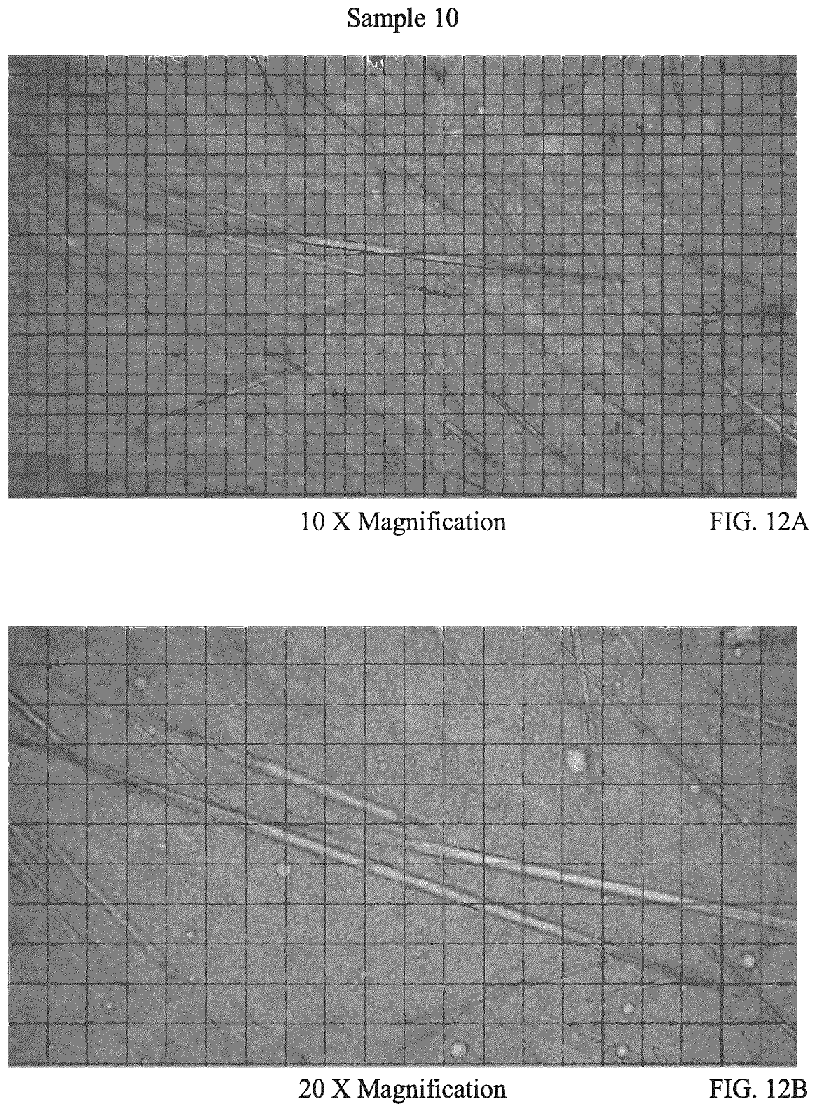

Those skilled in the art would understand that lecithin concentration may affect the formulation by changing the interaction between the aqueous and lipid phases at the boundaries between them. Typically, a higher lecithin ratio relative to the lipid phase concentration results in smaller non-continuous phase droplets. However, it may also be the case that the higher lecithin concentration may prevent the API crystals from aggregating. The higher lecithin concentration may also make the formulation very viscous. In the use case of cancer therapeutics, the higher viscosity may be acceptable as the delivery needle can be a large gauge needle for placement of the drug product. In this Example, five samples, samples 6-10, were developed and analyzed using differing concentrations of lecithin.
Sample 6 uses the base formulation but with the lecithin component being reduced to 0.5% and replaced with soybean oil. Images of the resulting material are shown under 10× magnification in FIG. 8A and under 20× magnification in FIG. 8B. The oil droplets are small, and the API crystals are long needle form. The emulsion remained stable for 3 days but can be re-suspended or re-emulsified into an acceptable emulsion through agitation, such as shaking. The sample forms a white opaque emulsion/suspension. Note that the large voids in FIGS. 8A and 8B are air pockets entrapped in the sample.
Sample 7 uses the base formulation but with the lecithin component being slightly reduced to 2.5% and replaced with soybean oil. Images of the resulting material are shown under 10× magnification in FIG. 9A and under 20× magnification in FIG. 9B. The oil droplets are small, and the API crystals are long needle form. The emulsion remained stable for 4 days but can be re-suspended or re-emulsified into an acceptable emulsion through agitation, such as shaking. The sample forms a white opaque emulsion/suspension. Note that the large voids in FIGS. 9A and 9B are air pockets entrapped in the sample.
Sample 8 uses the base formulation but with the lecithin component being increased to 5%, with soybean oil being removed to compensate. Images of the resulting material are shown under 10× magnification in FIG. 10A and under 20× magnification in FIG. 10B. The oil droplets are small, and the API crystals are long needle form. The emulsion remained stable for 9 days but can be re-suspended or re-emulsified into an acceptable emulsion through agitation, such as shaking. The sample forms a white opaque emulsion/suspension. Note that the large voids in FIGS. 10A and 10B are air pockets entrapped in the sample.
Sample 9 uses the base formulation but with the lecithin component being increased to 10%, with soybean oil being removed to compensate. Images of the resulting material are shown under 10× magnification in FIG. 11A and under 20× magnification in FIG. 11B. The oil droplets are very small, and the API crystals are long needle form. The crystals in the sample have aggregated into clumps. The emulsion remained stable for 23+ days. The sample forms a white opaque emulsion/suspension. Note that the large voids in FIGS. 11A and 11B are air pockets entrapped in the sample.
Sample 10 uses the base formulation but with the lecithin component being increased to 20%, with soybean oil being removed to compensate. Images of the resulting material are shown under 10× magnification in FIG. 12A and under 20× magnification in FIG. 12B. The oil droplets are very small, and the API crystals are long needle form. The crystals in the sample are separated from each other and show very little aggregation. The emulsion remained stable for 15 days. The sample forms a white opaque emulsion/suspension that is very viscous, such that it would be difficult to inject.

### Example 7 Aqueous Phase Thickening

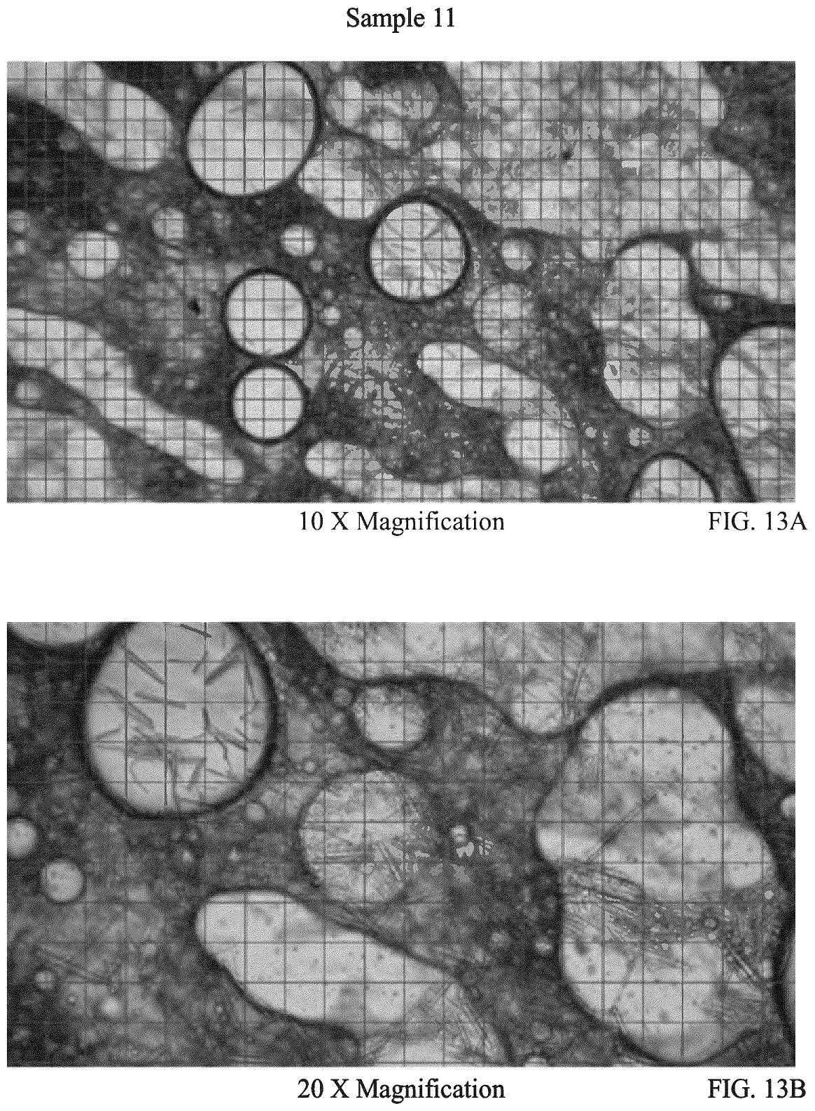

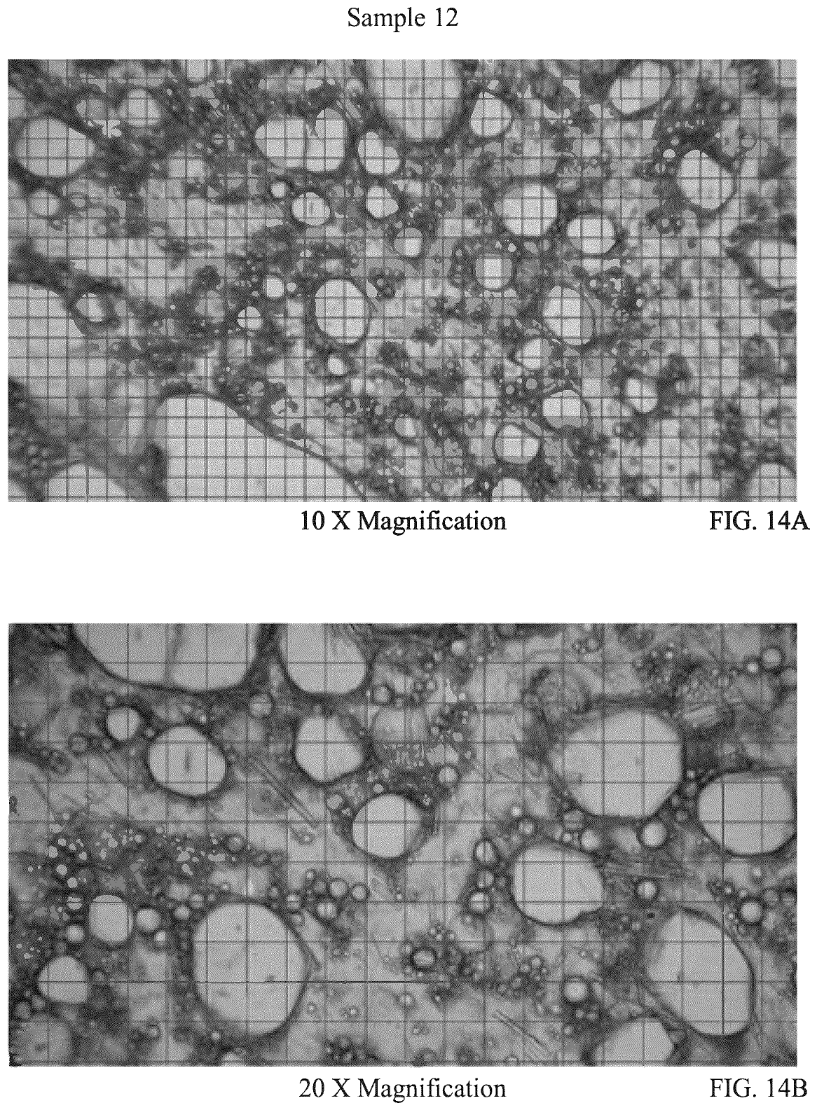

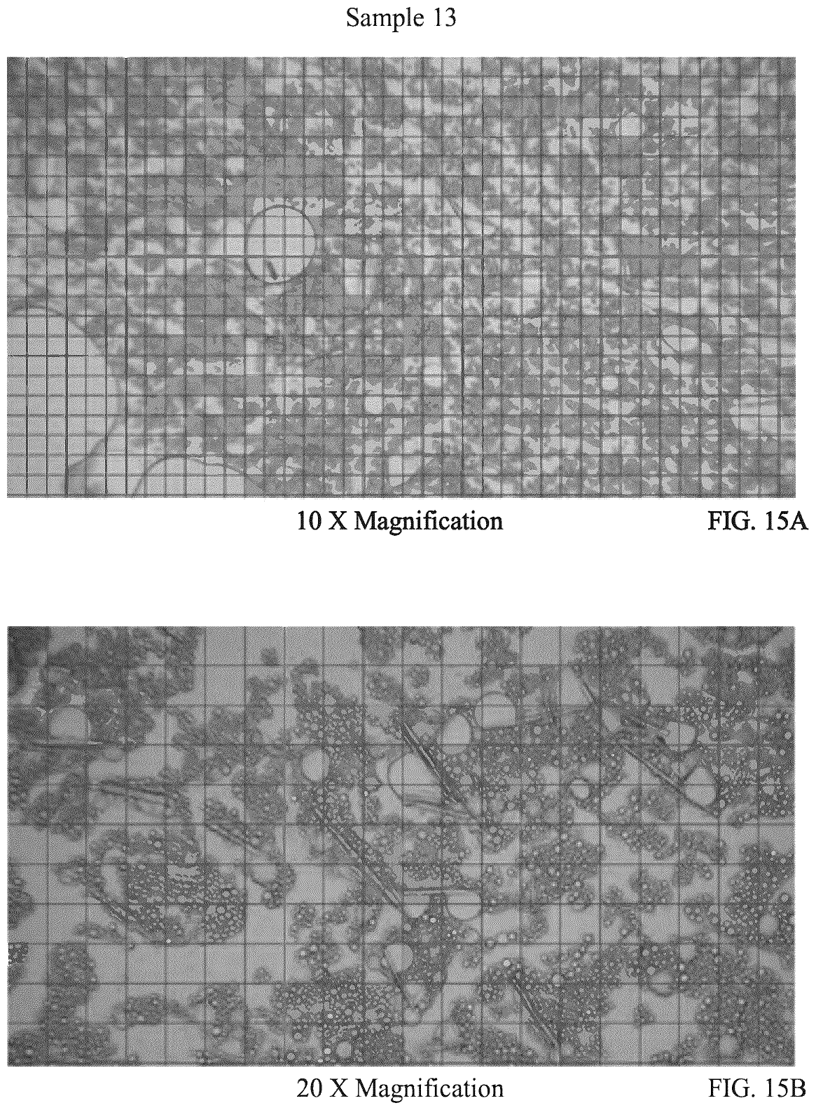

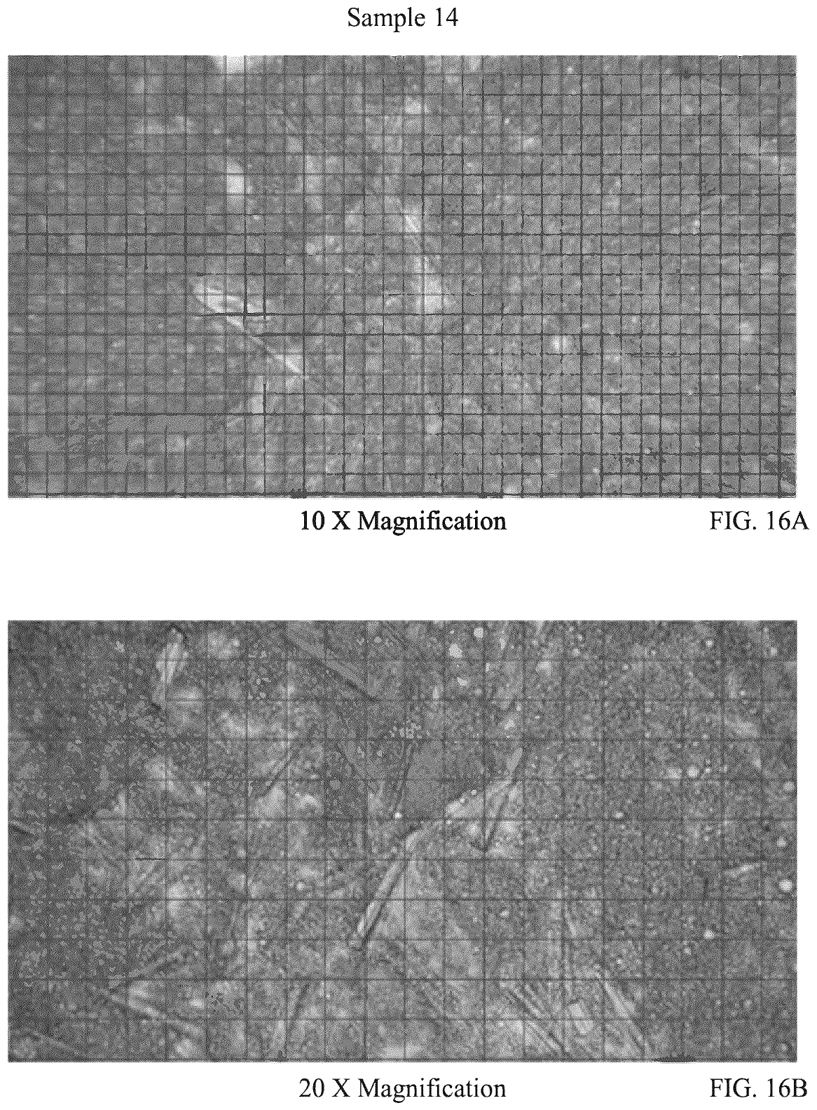

In this Example, sodium hyaluronate was used as a thickening agent for the aqueous phase to slow or impede the interaction between the lipid non continuous phase droplets and stabilize the emulsion. Hyaluronic acid, from sodium hyaluronate, may also act as an emulsifier, as the molecule has both polar and nonpolar moieties. In this Example, sodium hyaluronate concentration is measured relative to the aqueous phase, not the whole drug.
Sample 11 uses the base formulation but has no sodium hyaluronate in the aqueous phase. Images of the resulting material are shown under 10× magnification in FIG. 13A and under 20× magnification in FIG. 13B. The oil droplets are large, and the API crystals are long needle form. The emulsion of the sample broke quickly—within minutes—and separated into oil and aqueous phases.
Sample 12 uses the base formulation but has a sodium hyaluronate concentration of 0.1 percent in the aqueous phase. Images of the resulting material are shown under 10× magnification in FIG. 14A and under 20× magnification in FIG. 14B. The oil droplets are smaller than those of sample 11. The API crystals are long needle form. The emulsion remained stable for 2 days but can be re-suspended or re-emulsified into an acceptable emulsion through agitation, such as shaking.
Sample 13 uses the base formulation but has a sodium hyaluronate concentration of 0.15 percent in the aqueous phase. Images of the resulting material are shown under 10× magnification in FIG. 15A and under 20× magnification in FIG. 15B. The oil droplets are small and relatively uniform. The API crystals are long needle form. The emulsion showed increased stability over sample 11.
Sample 14 uses the base formulation but has a sodium hyaluronate concentration of 1 percent in the aqueous phase. Images of the resulting material are shown under 10× magnification in FIG. 16A and under 20× magnification in FIG. 16B. The oil droplets are small and relatively uniform. The API crystals are long needle form. The emulsion remained stable for 8 days.

As can be seen, at concentrations below 0.1 percent, the emulsions quickly broke and separated. At concentrations above 0.1 percent, the emulsions became progressively more stable.

### Example 8 Addition of Glycerol

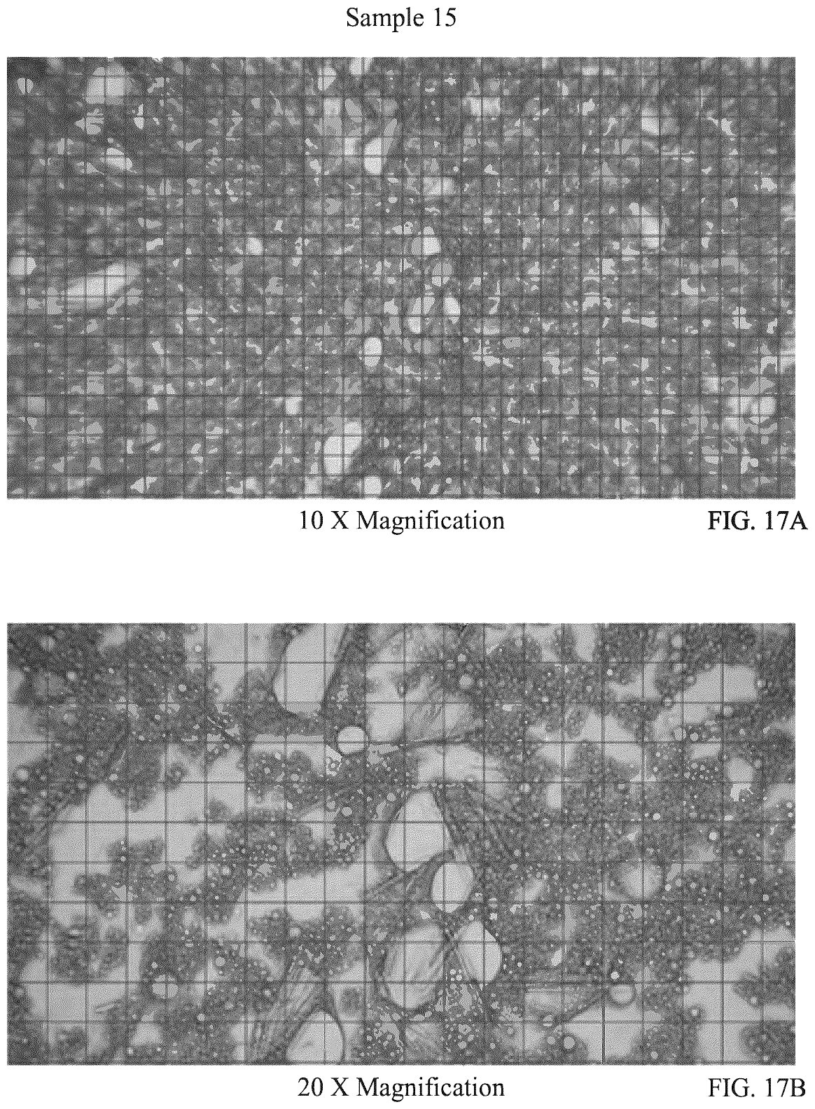

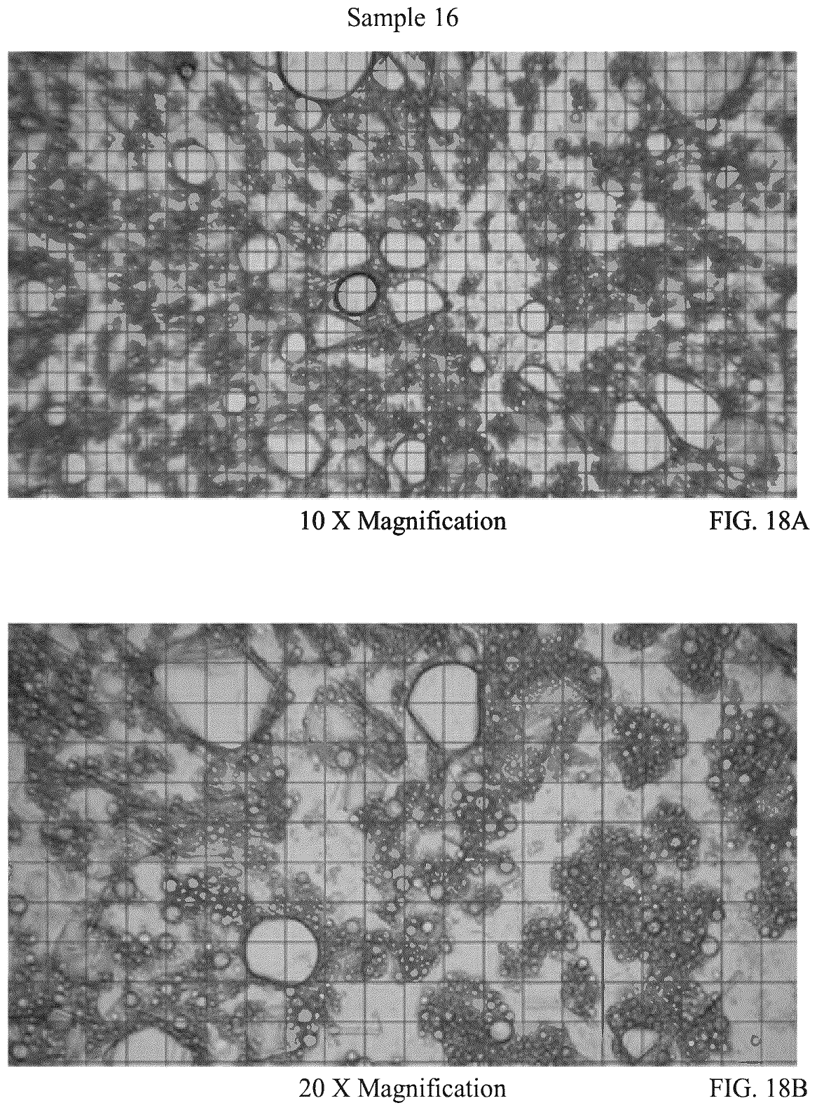

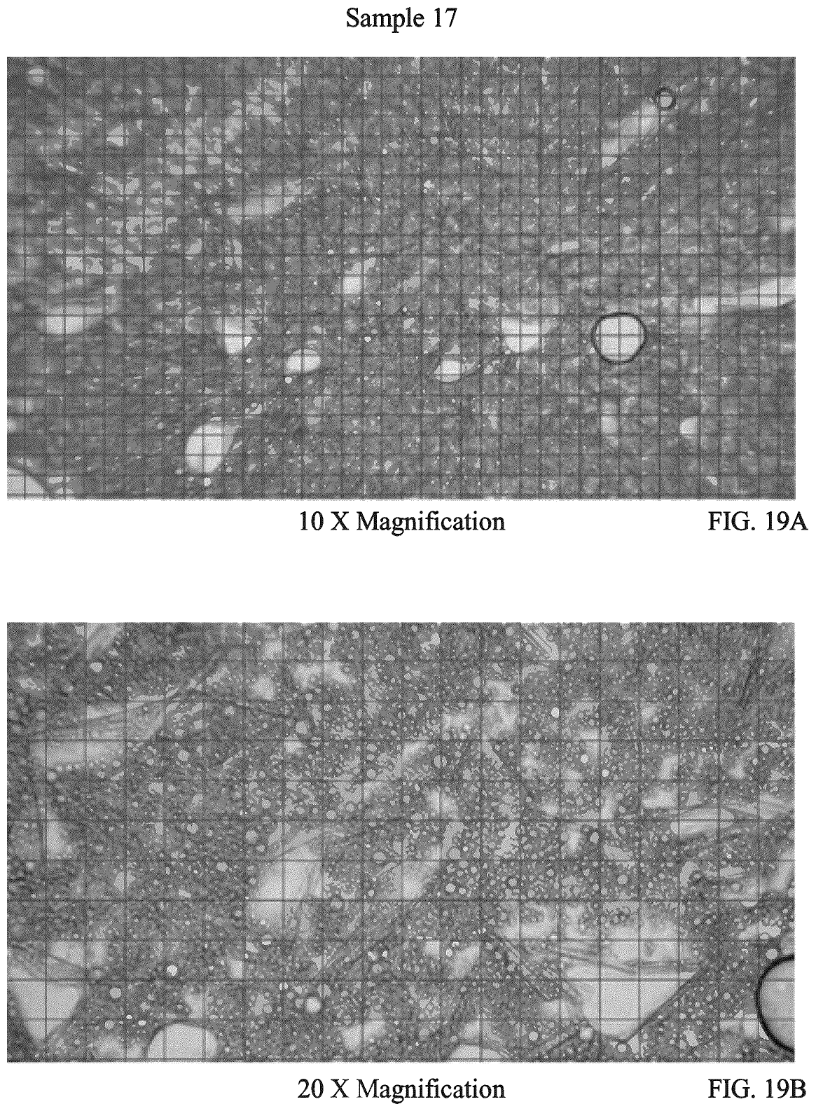

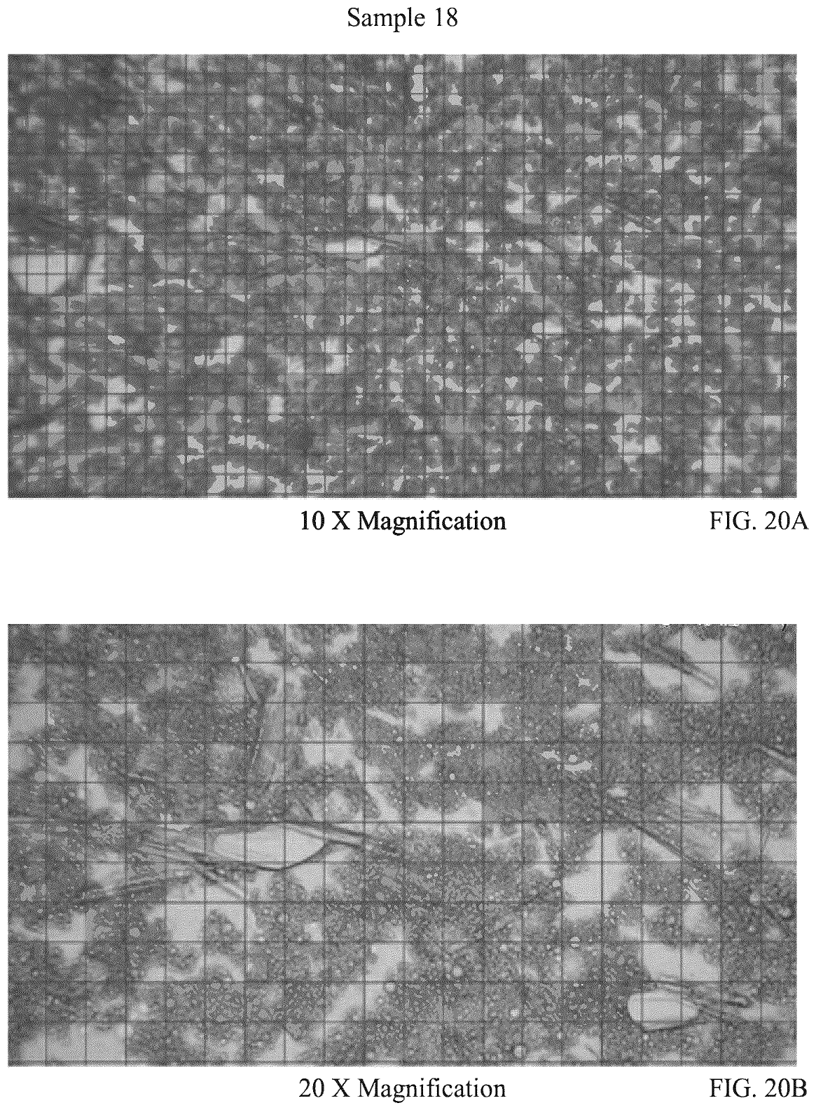

In this Example, glycerol was added as an aqueous phase modifier for the aqueous phase, which tended to increase the viscosity of the aqueous phase. Glycerol concentration in this Example is measured relative to the whole formulation, rather than any particular phase.
Sample 15 uses the base formulation but has a glycerol concentration of 1.7% relative to the whole formulation. Images of the resulting material are shown under 10× magnification in FIG. 17A and under 20× magnification in FIG. 17B The oil droplets are small and relatively uniform. The API crystals are long needle form. The emulsion remained stable for 1 day.
Sample 16 uses the base formulation but has a glycerol concentration of 3.0% relative to the whole formulation. Images of the resulting material are shown under 10× magnification in FIG. 18A and under 20× magnification in FIG. 19B. The oil droplets are small and relatively uniform. The API crystals are long needle form. The emulsion remained stable for 1 day.
Sample 17 uses the base formulation but has a glycerol concentration of 5.0% relative to the whole formulation. Images of the resulting material are shown under 10× magnification in FIG. 19A and under 20× magnification in FIG. 19B. The oil droplets are small and relatively uniform. The API crystals are long needle form. The emulsion remained stable for 1 day.
Sample 18 uses the base formulation but has a glycerol concentration of 10% relative to the whole formulation. Images of the resulting material are shown under 10× magnification in FIG. 20A and under 20× magnification in FIG. 20B. The oil droplets are smaller and more uniform than samples 18, 19, and 20. The API crystals are long needle form. The emulsion remained stable for 1 day.

As sample 18 showed improvements in oil drop size and consistency, a positive correlation between the concentration of glycerol or a similar solvent and the droplet consistency. A negative correlation can be seen between glycerol/solvent concentration and droplet size.

### Example 9 Lipid Phase Thickening

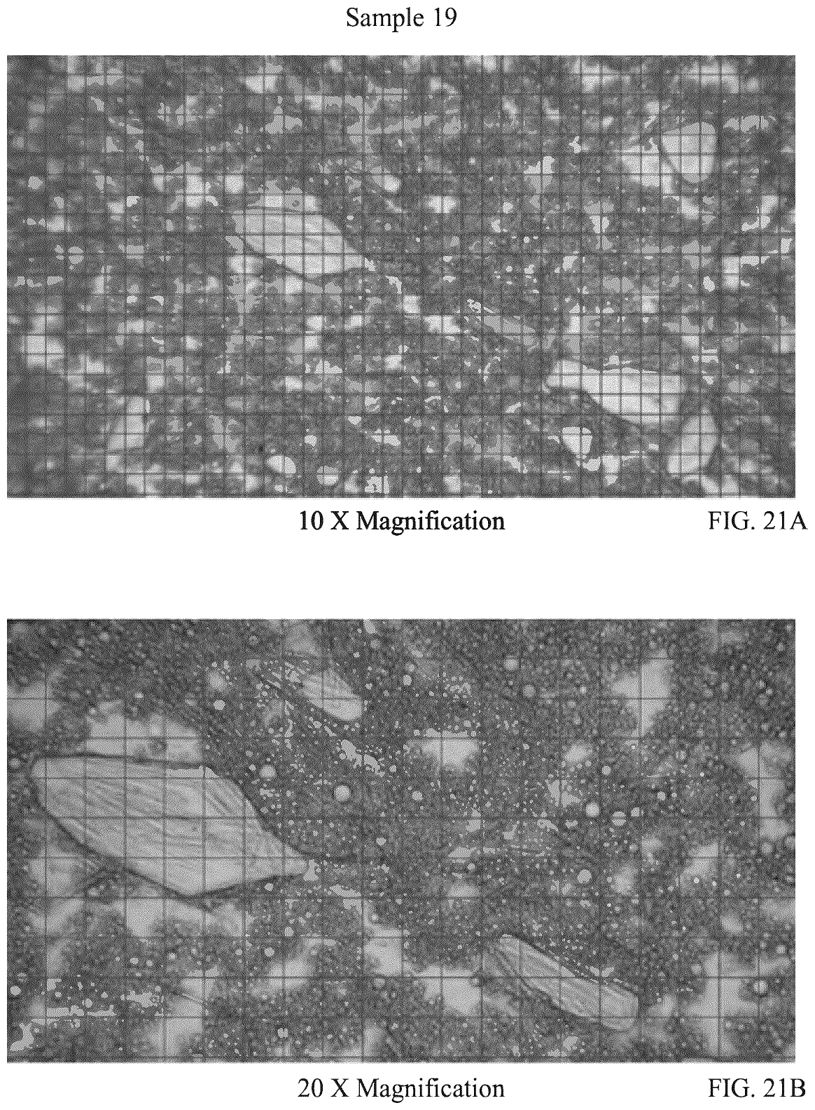

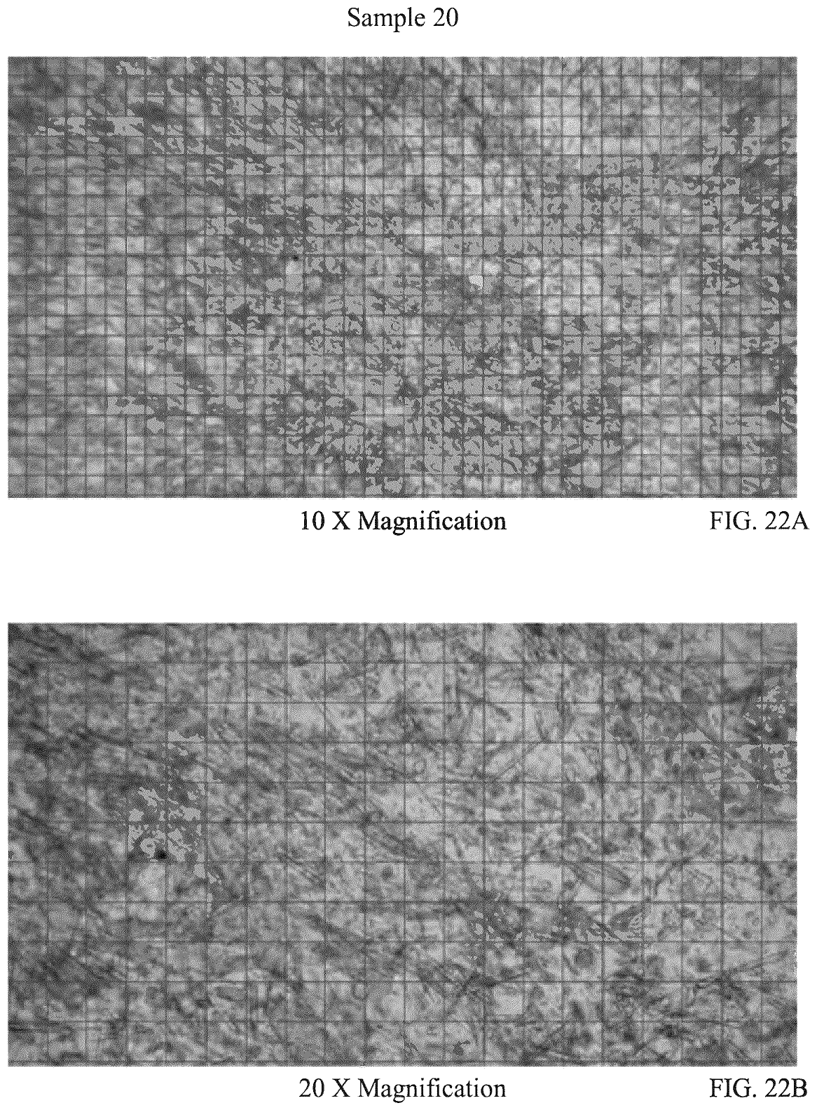

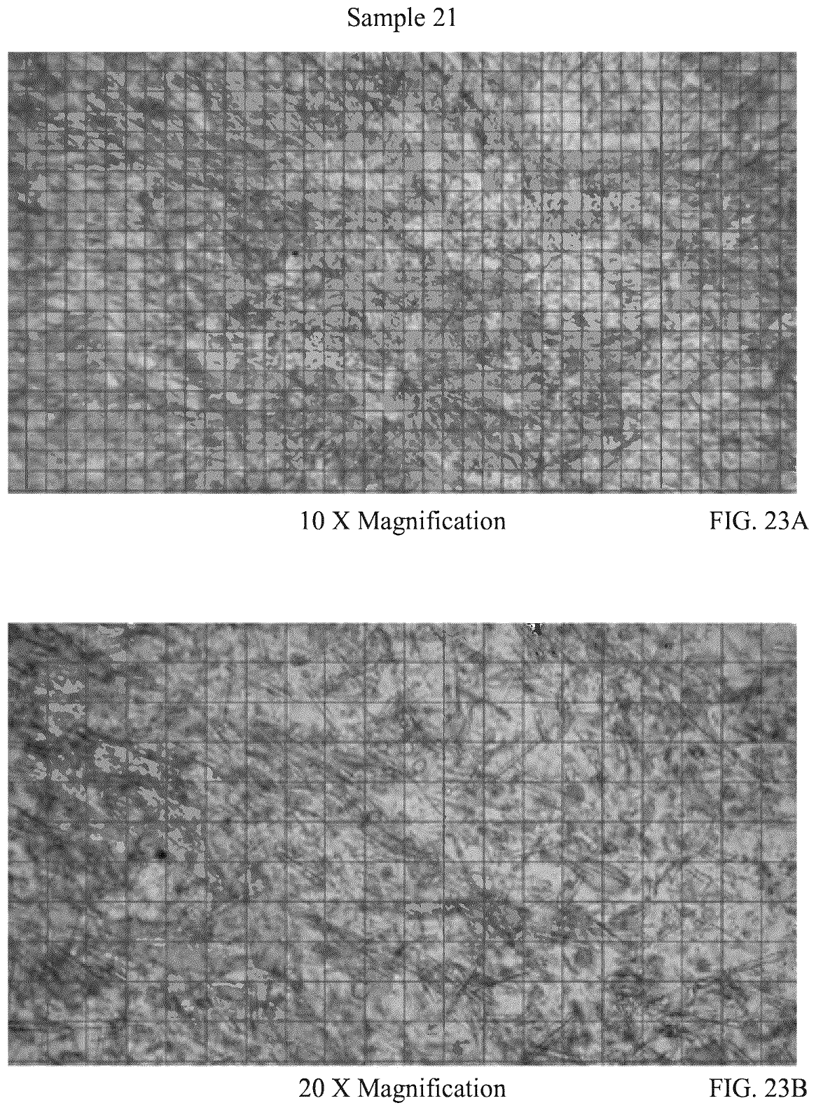

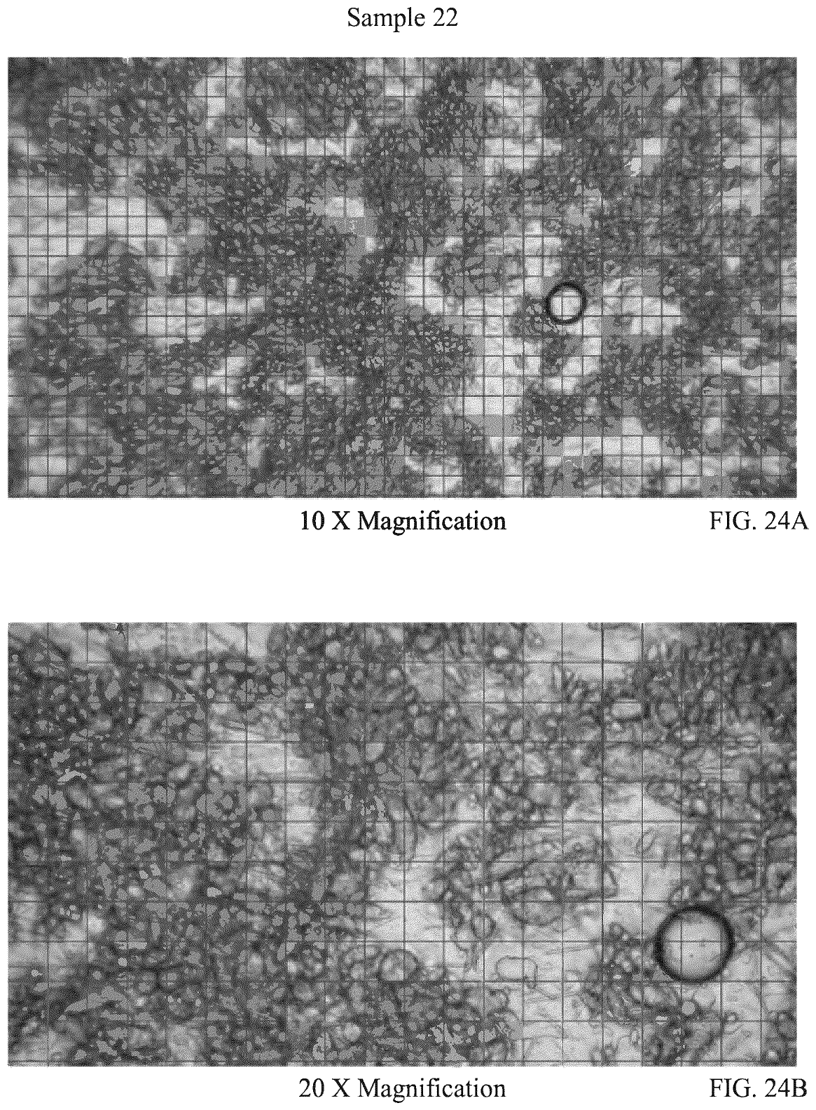

In this Example, either coconut oil or carnauba wax was added to the lipid phase of the formulation to increase its viscosity. As the thickening agent, coconut oil or carnauba wax, is added to the formulation, the added mass of soy oil was equally reduced to maintain the total amount of lipid phase in the formulation. Thickening agent concentrations in this Example are measured relative to the whole formulation, rather than any particular phase.
Sample 19 uses the base formulation but with 10% of the formulation consisting of coconut oil and an equal reduction in soy oil. Images of the resulting material are shown under 10× magnification in FIG. 21A and under 20× magnification in FIG. 21B. The oil droplets are generally small, but an increase in the size and number of large droplets can be observed. The API crystals are long needle form. The product was a stable emulsion.
Sample 20 uses the base formulation but with 1.0% of the formulation consisting of carnauba wax and an equal reduction in soy oil. Images of the resulting material are shown under 10× magnification in FIG. 22A and under 20× magnification in FIG. 22B. The API crystals are long needle form, and the wax and lipids tend to coat the crystals. The product was a stable emulsion.
Sample 21 uses the base formulation but with 2.5% of the formulation consisting of carnauba wax and an equal reduction in soy oil. Images of the resulting material are shown under 10× magnification in FIG. 23A and under 20× magnification in FIG. 23B. The API crystals are long needle form. The wax and lipids coating on the crystals is thicker than seen in sample 20. The product was a stable emulsion.
Sample 22 uses the base formulation but with 5.0% of the formulation consisting of carnauba wax and an equal reduction in soy oil. Images of the resulting material are shown under 10× magnification in FIG. 24A and under 20× magnification in FIG. 24B. The API crystals are long needle form. The wax and lipids have coated the crystals and have formed microparticles separate from the crystals. The product was a stable emulsion.

The carnauba wax appeared to coat much of the surface of the crystals. In higher concentrations, such as 5%, the carnauba wax also formed microparticles apart from the crystals. As would be understood, this tendency for the wax and lipids to coat the API crystals can lead to an increased barrier to diffusion of the API from the formulation into the subject. This increased barrier to diffusion can lead to a slower, more consistent release of the API into the subject. A slower more consistent release of the API allows for more of the formulation to be administered at a time, due to the lower rate of introduction to the subject.

### Example 10 Continuous Lipid Phase

In some implementations, and in this Example, it may be desirable to disperse the aqueous phase into a continuous lipid phase, rather than dispersing a lipid phase into a continuous aqueous phase, as in the other Examples. Having a continuous lipid phase may be beneficial when the API in use is hydrophilic, rather than hydrophobic. The formulation for sample 23 consisted of 60% lipid phase and 40% aqueous phase, by volume, with components of each phase kept in relative proportion, except where noted. The sodium hyaluronate in the formulation was 0.15% by weight, relative to the aqueous phase.

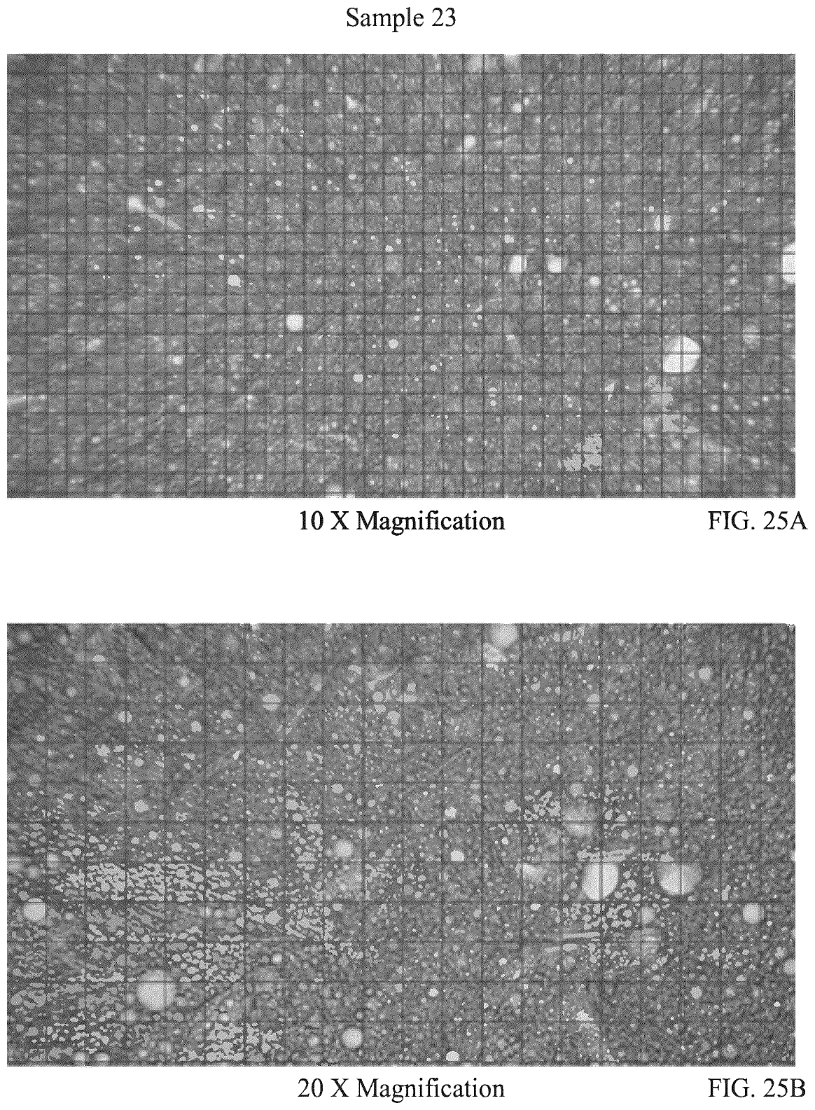

Images of the resulting material are shown under 10× magnification in FIG. 25A and under 20× magnification in FIG. 25B. The API crystals are long needle form. The aqueous phase droplets were varied in size.

### Example 11 Increased Mixing Energy

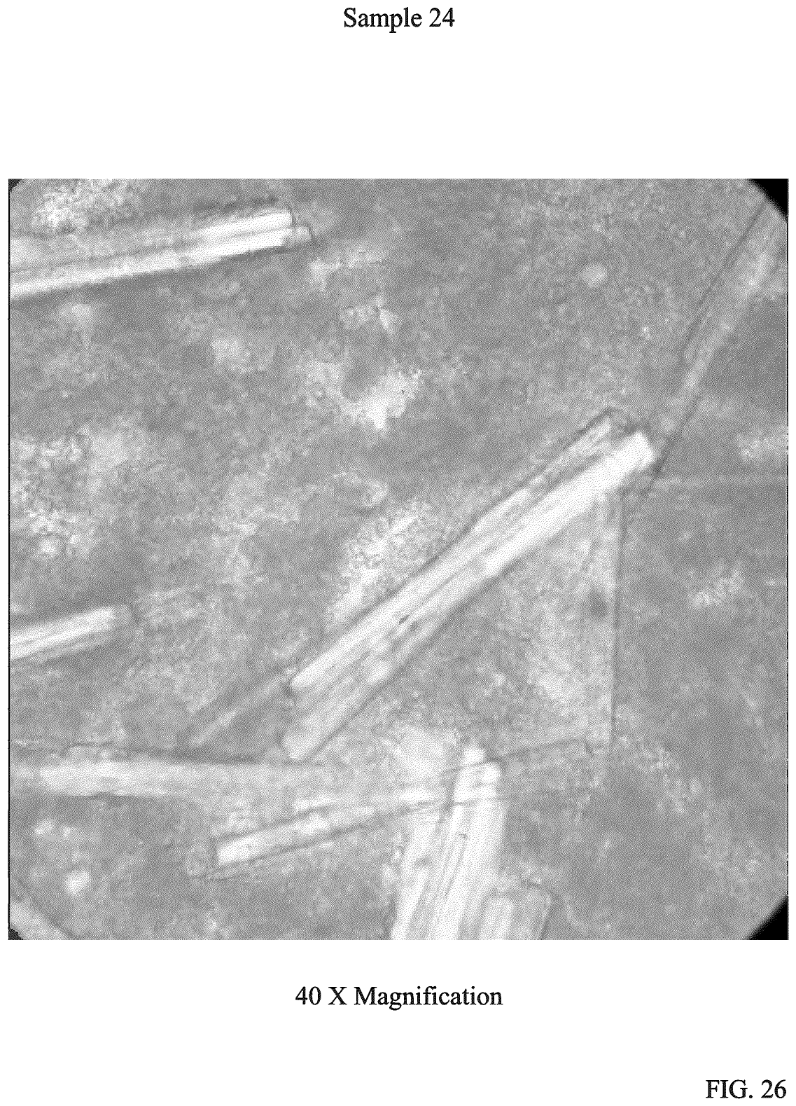

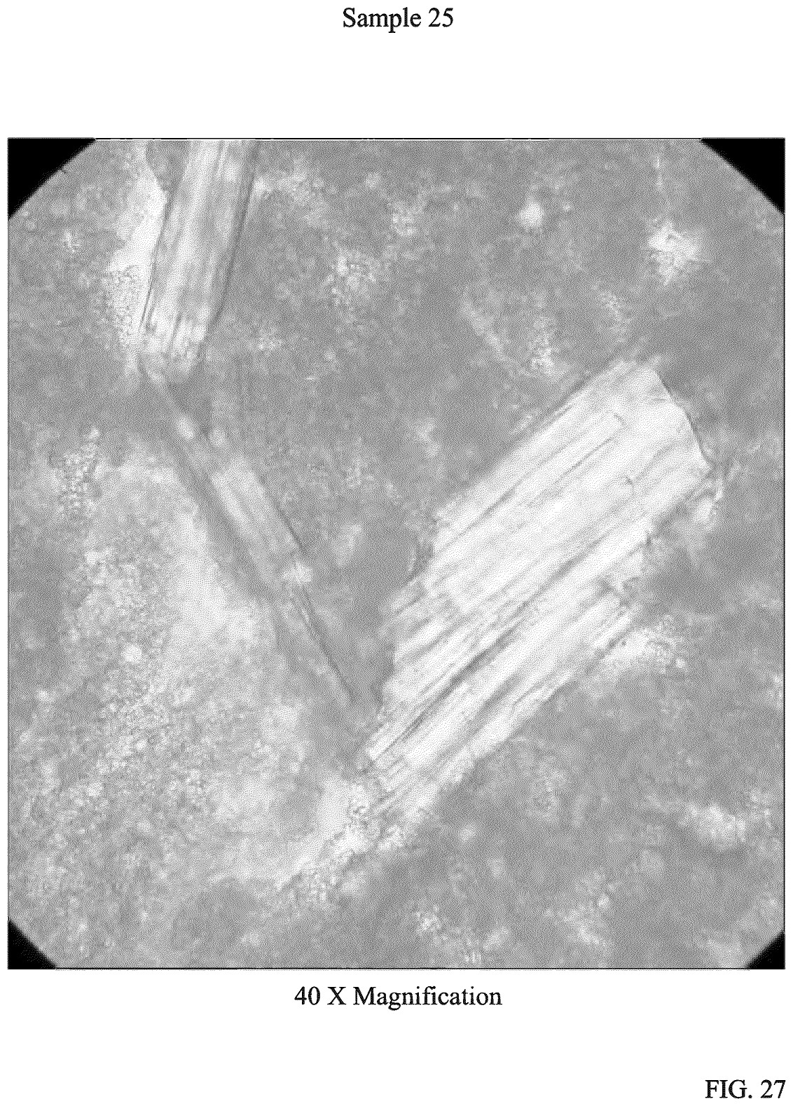

Sample 24 uses the base formulation but used more aggressive mixing energy. This was achieved by using a ¾ horsepower Silverson high-shear rotor-stator homogenizer at 6000 RPM. An image of the resulting material is shown under 40× magnification in FIG. 26. The oil droplets are smaller than those found in formulations made with standard mixing energy. The smaller oil droplets tend to coat the surface of the API crystals. The emulsion remained stable for more than 7 months.
Sample 25 uses the base formulation but used the ¾ horsepower Silverson high-shear rotor-stator homogenizer at 12,000 RPM. An image of the resulting material is shown under 40× magnification in FIG. 27. API crystal aggregations formed as a result of the higher mixing energy. These aggregations remain stable and do not separate. The increased mixing energy also increased the size distribution of the API crystals and crystal agglomerates.

### Example 12-Increased CA Loading

In a comparison between a formulation containing no CA (placebo) and a formulation containing CA (active), it was observed that the placebo formulation had broken, while the active formulation had not. However, samples with higher CA concentrations, in the form of ropivacaine, were less stable than those of lower concentrations. A sample with 29.34 mg/mL ropivacaine had greater stability than a sample with 38.51 mg/mL ropivacaine, which had greater stability than a sample with 47.69 mg/mL ropivacaine, which had greater stability than placebo. The 29.34 mg/mL ropivacaine sample is stable for at least 7 months.

Although the disclosure has been described with reference to preferred embodiments, persons skilled in the art will recognize that changes may be made in form and detail without departing from the spirit and scope of the disclosed apparatus, systems and methods.

What is claims is:

1. A composition for treating a tumor in a subject in need thereof, comprising:
an emulsion comprising:
an aqueous carrier; and liquid lipid phase dispersed into droplets within the aqueous carrier, and a first chemotherapeutic agent within the lipid phase.

2. The composition of claim 1, wherein the first chemotherapeutic agent comprises a first plurality of chemotherapeutic agent crystals.

3. The composition of claim 1, wherein the first chemotherapeutic agent is dissolved with the lipid phase.

4. The composition of claim 2, further comprising a second plurality of chemotherapeutic agent crystals within the aqueous carrier, but not the lipid phase and wherein the second plurality of chemotherapeutic agent crystals dissolves and elutes from the emulsion at a faster rate than the first plurality chemotherapeutic agent crystals.

5. The composition of claim 1, wherein the composition further comprises one or more additional chemotherapeutic agents, different from the first chemotherapeutic agent.

6. The composition of claim 1, wherein the lipid phase comprises a triglyceride.

7. The composition of claim 1, wherein the aqueous carrier further comprises an emulsifier and a polyol wherein the emulsifier is tyramine substituted hyaluronic acid and wherein the polyol is glycerol and is present in an amount of from about 0.25 to about 2.5% (w/v) of the composition.

8. The composition of claim 1, wherein the lipid phase further comprises a phospholipid present in an amount from about 0.1% to about 2.0% of the lipid phase.

9. The composition of claim 1, wherein the lipid phase is from about 10% to about 40% (w/v).

10. The composition of claim 1, wherein the stable emulsion further comprises lecithin, and wherein lecithin is present in amount of from 0.1-5% (w/v) of the emulsion.

11. The composition of claim 1, wherein the emulsion further comprises dextrose, and wherein dextrose is present in amount of from about 1-2% (w/v) of the emulsion.

12. The composition of claim 1, wherein the emulsion further comprises sorbitol, and wherein sorbitol is present in amount of from about 0.1-2% (w/v) of the emulsion.

13. The composition of claim 1, wherein the lipid phase comprises soybean oil in an amount from about 15% to 80% (v/v) and one or more medium chain triglycerides in amount from about 15% to about 20% (v/v).

14. The composition of claim 13, wherein the chemotherapeutic agent is present in an amount from about 0.1 mg/g soybean oil to about 300 mg/g soybean oil.

15. The composition of claim 1, wherein the chemotherapeutic agent is selected from anthracyclines, mTOR inhibitors, VEGF-TKI agents, and immune stimulators.

16. The composition of claim 1, wherein the lipid phase comprises tributyrate and/or stearate.

17. The composition of claim 2, wherein the lipid phase further comprises an oil and/or wax that is solid at 25° C. in an amount of from about 5% to about 30% of the lipid phase (w/w) and wherein the oil and/or wax coats the chemotherapeutic agent crystals.

18. The composition of claim 1, wherein the emulsion is stable for at least six months.

19. The composition of claim 1, wherein the chemotherapeutic agent is docetaxel and/or doxorubicin.

20. A composition for treating a tumor in a subject in need thereof, comprising:
an emulsion comprising:
a lipid carrier phase; and an aqueous phase dispersed into droplets within the lipid carrier phase, and a first chemotherapeutic agent within the aqueous phase.

21. The composition of claim 20, wherein the first chemotherapeutic agent comprises a first plurality of chemotherapeutic agent crystals.

22. The composition of claim 21, further comprising a second plurality of chemotherapeutic agent crystals present within the lipid carrier, but not the aqueous phase, and wherein the second plurality of chemotherapeutic agent crystals dissolves and elutes from the emulsion at a faster rate than the first plurality chemotherapeutic agent crystals.

23. The composition of claim 22, wherein the chemotherapeutic agent is hydrophilic and is dissolved within the aqueous phase.

24. The composition of claim 20, wherein the composition further comprises one or more additional chemotherapeutic agents, different from the first chemotherapeutic agent.

25. A method of treating a tumor in a subject in need thereof comprising administering to the subject and effective amount of a composition comprising:
an emulsion comprising:
an aqueous carrier; and lipid phase dispersed into droplets within the aqueous carrier, and
a first chemotherapeutic agent within the lipid phase and wherein the chemotherapeutic agent is eluted from the composition over a period of between about 4 and about 7 days.

26. The method of claim 25, wherein the chemotherapeutic agent comprises a plurality of chemotherapeutic agent crystals, and wherein the composition further comprises a second plurality of chemotherapeutic agent crystals within the aqueous carrier, but not the lipid phase and wherein the second plurality of chemotherapeutic agent crystals dissolves and elutes from the emulsion at a faster rate than the first plurality chemotherapeutic agent crystals.

27. The method of claim 25, wherein the composition further comprises an immune stimulator.
